SEQUENCE LISTING (16 sequences)

SEQ ID NO: 1 - DNA, 2888 bp, Homo sapiens
gtggcggcggaggcggcggaggccagggaggaagatgtcgtaatgagcgatccacagaccagcatggctgccactgctgctgtgagtcccagtgactacctgcagcctgccgcctccaccacccaggactcccagccatctcccttagccctgcttgctgcaacatgtagcaaaattggccctccagcagttgaagctgctgtgacacctcctgctcccccacagcccacaccgcggaaacttgtccctatcaaacctgcccctctccctctcagccccggcaagaatagctttggaatcttgtcctccaaaggaaatatacttcagattcaggggtcacaactgagcgcctcctatcctggagggcagctggtgttcgctatccagaatcccaccatgatcaacaaagggacccgatcaaatgccaatatccagtaccaggcggtccctcagattcaggcaagcaattcccaaaccatccaagtacagcccaatctcaccaaccagatccagatcatccctggcaccaaccaagccatcatcaccccctcaccgtccagtcacaagcctgtccccatcaagccagcccccatccagaagtcgagtacgaccaccacccccgtgcagagcggggccaatgtggtgaagttgacaggtgggggcggcaatgtgacgctcactctgcccgtcaacaacctcgtgaacgccagtgacaccggggcccctactcagctcctcactgaaagccccccaaccccgctgtctaagactaacaagaaagcaaggaagaagagccttcctgcctcccagccccctgtggctgtggctgagcaggtggagacggtgctgatcgagaccaccgcggacaacatcatccaggcaggaaataacctgctcattgttcagagccctggtgggggccagccagctgtggtccagcaggtccaggtggtgccccccaaggccgagcagcagcaggtggtacagatcccccagcaggctctgcgggtggtgcaggcggcatctgccaccctccccactgtaccccagaagccctcccagaactttcagatccaggcagctgagccgacacctactcaggtctacatccgcacgccttccggtgaggtgcagacagtccttgtccaggacagccccccagcaacagctgcagccacctctaacaccacctgtagcagccctgcatcccgtgctccccatctgagtgggaccagcaaaaagcactcagctgcaattctccgaaaagagcgtcccctgccaaagattgccccagccgggagcatcatcagcctgaatgcagcccagttggcggcagctgcccaggcaatgcagaccatcaacatcaatggtgtccaggtccagggcgtgcctgtcaccatcaccaacacaggcgggcagcagcagctgacagtgcagaatgtttctgggaacaacctgaccatcagtgggctgagccccacccagatccagctgcaaatggaacaagccctggccggagagacccagcccggggagaagcggcgccgcatggcctgcacgtgtcccaactgcaaggatggggagaagaggtctggagagcagggcaagaagaagcacgtgtgccacatccccgactgtggcaagacgttccgtaagacgtccttgctgcgtgcccatgtgcgcctgcacactggcgagcggccctttgtctgcaactggttcttctgtgggaagaggttcacacggagtgacgagctccaacggcatgctcgcacccacacaggggacaaacgcttcgagtgcgcccagtgtcagaagcgcttcatgaggagtgaccacctcaccaagcattacaagacccacctggtcacgaagaacttgtaaggccaactgcggcgggaggccctgaagatgcagtcccccacctgtgtcctccctgggcccctggtggaaaggagccctgtggctgccttgggcctgccctcagccccactcctgttctgcaactgtccccacaggaaggggctctgttccctgtattgtcctccttctgaagccccttggctctgccttggcccttcccctcaccacgagctcccggcctgcccagactgtggacactggccgtgcccaatgagacgttctaaaccaggacgcgtgggaacccttatttccaaaggaaaaacatgcatttcactccgtcgaggagcaaagtgagcccctaccccccaccccgatccccgctcccaacactgccggagtcgcgtcatgccatgccccctctcctgcacctccctggccctgccggccactgtggacgccctggggcttggcacccacctctggagaaactcggggccacctccactccatgtgcccagccccgccacaacctctcctccagcacattccagctctatttaaaaagtaaagacacccaccgactcctgatccccctctttttctatggagaacgttgccttatactctctacttcagatgatgaacactgtgtactgtgtgtgctttaaagaagttttatttaattgctcccttcttcctttccttgttattcacctccctgatgcctgctttcagttgagggttgggggcaatgatgagcatatgaattttttctcactctagcaattcccttttctaaatgacacagcatttaaactcaaatctggattcagataacagcacctgcacatcctgcacctcctccctctcccttcacctcacccctgcccggcccaagctctacttgtgtacagtgtatattgtataatagacaattgtgtctactacatgtttaaaaacacattgcttgttatttttgaggcttttaaattaaacaaaaatccaactttaaaaaaaaaaaaaaa

SEQ ID NO: 2 - DNA, 999 bp, Homo sapiens
cccgcgtcggtgcccgcgcccctccccgggccccgccatgggcctcaccgtgtccgcgctcttttcgcggatcttcgggaagaagcagatgcggattctcatggttggcttggatgcggctggcaagaccacaatcctgtacaaactgaagttgggggagattgtcaccaccatcccaaccataggcttcaatgtagaaacagtggaatataagaacatctgtttcacagtctgggacgtgggaggccaggacaagattcggcctctgtggcggcactacttccagaacactcagggcctcatctttgtggtggacagtaatgaccgggagcgggtccaagaatctgctgatgaactccagaagatgctgcaggaggacgagctgcgggatgcagtgctgctggtatttgccaacaagcaggacatgcccaacgccatgcccgtgagcgagctgactgacaagctggggctacagcacttacgcagccgcacgtggtatgtccaggccacctgtgccacccaaggcacaggtctgtacgatggtctggactggctgtcccacgagctgtcaaagcgctaaccagccaggggcaggcccctgatgcccggaagctcctgcgtgcatccccggatgaccatactcccggactcctcaggcagtgccctttcctcccacttttcctcccccatagccacaggcctctgctcctgctcctgcctgcatgttctctctgttgttggagcctggagccttgctctctgggcacagaggggtccactctcctgcctgctgggacctatggaaggggcttcctggccaaggccccctcttccagaggaggagcagggatctgggtttccttttttttttctgttttgggtgtactctaggggccaggttgggagggggaaggtgagggcttcgggtggtgctataatgtggcactggatcttgagtaataaatttgctgtggtttgaaaaaaaaaaaaaaaaaaaaa

SEQ ID NO: 3 - DNA, 3487 bp, Homo sapiens
gtggcggtggctgcggcgacggcagaggcgaagggagccggatcgccgacctgagcgggaggcggcggtggcggccatggcggcagatggagagcgttccccgctgctgtctgagcccatcgacggtggcgcgggcggcaacggtttagtggggcccggcgggagtggggctgggcccgggggaggcctgaccccctccgcaccaccgtacggagccggtaaacatgccccgccccagggtaagccggggcgggtccgaggtgctccccggggtactctgaaagccggggagggggcgggaccgagggcggaggcgggtcccagtcgccaggtgcgggactgctgcacctgtgactgggcgaggcttccttccctccgtaatcgcgaccacagcctagggacggaagggggttctgagcaacctgatagaagtgccaattatgagaagccctccgagcttggtcagagggttgaagatcagaaggacttccctaccaccgtggagcatcagtgggggtgtaagtgatcccagcccttctatttgcttcctctccagcatttcccccgtttcccgaggggcatccagccgtgttgcctggggaggacccacccccctattcacccttaactagcccggacagtgggagtgcccctatgatcacctgccgagtctgccaatctctcatcaacgtggaaggcaagatgcatcagcatgtagtcaaatgtggtgtctgcaatgaagccaccgtgagttacacatatctatgaaatgggccctgtttcctggatcctctttctgatgtcttggttctagaccctgaccttccggctattagccaagtgcttttgatgatacccaggtttcagttccaggtgtctcacacagccatttccccagaagccactcaccaaagctaatgttcactttctctcacttttacacctagcctagttcctatttgcaaatctcatgatatagtctttcttttatttctccttcctggttagcaccttatttttctgatctcataaagtgtttttggagggaagtggaggggattgggattagaggtttgcttgctgatgaccctattattctctagccaatcaagaatgcacccccagggaaaaaatatgttcgatgcccctgtaactgtctccttatctgcaaagtgacatcccaacggattgcatgccctcgtccctactggtaagaggcataaggtggggaagggcctaagtggggaactggaaagtcaaaaaaggatgagcgtatacagagaatgtaaaggtgagagagcctagtgtttatttaggagaaaaggctttgaagcatgtgcctcaggaatgttatagctgtctttctcgtttctcaataaaaatattgagatgaaatgatgtcgtttcggagaatagagagccttggggactgggtgtgttatcctgaggtcggaggggaattggggacctgaagtttaaacagtgctctttctttctcaaggattcttgagggtatacagttgggggacagagtatcttaagtacagagaagtcgagtgacttaatagacagggagtgggggatgtggaacagggactgtgaagatttttaggattaaaaatttttcaaacacaagtttgaaaatacaagtctttttcttttgtatagcaaaagaatcatcaacctggggcctgtgcatcccggacctctgagtccagaaccccaacccatgggtgtcagggttatctgtggacattgcaagaatacttttctggtgaggaaggggtattgggaaggggaggggaaaggagactaagagtcatttcgagtatatttcttagagtaatggtaatgacccctgaaaggtctgtcctatgggaacatgttctgcatccccaccccaaggttctcattgagggagaccctgcttgtgctattatttttgttttctttctccatagtggacagagttcacagaccgcactttggcacgttgtcctcactgcaggaaagtgtcatctattgggcgcagatacccacgtaagagatgtatctgctgcttcttgcttggcttgcttttggcagtcactgccactggccttgccgtgagtacccttgccccaacctctttcattctgcagcctcatctccataggctaagatttgggaaactgctaccctaaaaaaaagtggaagaaacttaggggactagtttgttttgttttaagatatggatgagctaaagtgcaaagtggctgatcaaacagactttattactactacaagagtgaaaaacagccttcctttctctgtaggatgaggataggacagtgaaattcttaatttaagagttgctatttttcaaacctggctcagttgtcagatattaagaaaaactgagatacagtgtgggatgggatgagtatgttacgcctaagggaaggaagctgatcagctctgcctttaagaaggtccctgagggtggctacatgtggataaggaacaaggactgaagcgtgagttattactgttcttagaactaataggaggtagtggagaccaacattaaccccatctttcttttcttctccctccttatcttcatcagtttggcacatggaagcatgcacggcgatatggaggcatctatgcagcctgggcatttgtcatcctgttggctgtgctgtgtttgggccgggctctttattgggcctgtatgaaggtcagccaccctgtccagaacttctcctgagcctgatgacccacagactgtgcctggcccctccctggtggggacagtgacactacgaagggagctggggtagttaaaggctcccggggcttctagaaggaagccaagcagctgccttccttttccctggggagaggtaggaaggaaccaggccctcacttaggtttggaggggcagataagagcactgctgaccatctgctttcctccaagggttgctgtgtctagggtgaagtaggcaaaacgttgcccttaaaactgggccctgaagacggttccagccttgtccttcctgtgtgctccctgagagccattcctgtcccttacacattccagggcagggtgggggtgggtagccctgggggttcccctccctcttgtgcaccattaggactttgctgctgctattgcacttcaccagaggttggctctggcctcagtaccctcagtctcctctccccacattgtgtcctgtgggggtggggtcagccgctgctctgtacagaaccacaggaactgatgtgtatataactatttaatgtgggatatgttcccctattcctgtatttcccttaattcctcctcccgaccttttttacccccccagttgcagtatttaactgggctgggtagggttgctcagtctttgggggaggttagggacttatcctgtgcttgtaaataaataaggtcatgactctaaaaaaaaaaaaaaaaaaaaaaaaaaaaaaaaaaa

SEQ ID NO: 4 - DNA, 1651 bp, Homo sapiens
ctgtcagcacggggcctggcatgtaattggtctgcacccactggtgcactgaactgccataacctcaggttttctttcttgctgatacccctgggtcatgttctttggcaaataacatgattcattatgaagtagagttcagcaaaggacaaggatgaaagttgtcatttagagaactgccattcagactttcttgtctaggtaaagagcaaggtcttctctcttttcaactcattttctaaatttaaactgacgatgagaatatggatgatgtgtagcttccttctcccccactgatttttggttcaggctctgggtttttggcaagaacttacagatctcacttattattggccacccttctgctttaagacctgtcagggcttgtctgaaataaaactggaagcacttctgattccatcctcactgctttcctccttcaccgtcagacagcattactgtatagcactgagtgaggggccctgacactggaaggtggcaggtggggcctggccgccagtgaggtatcatcatttgtgtgtgctcatgtgtgcgttgggcttgttgtatctgaggcatgaacattccatatacacggcttaaagagttttcttcccataccgaaagcatatattcggagaggacccaacttattcagcatagccttgttcccatagtagccatcctattcccccacagcctctactttaggaaagctccccgtccccatatgaaatccaaaccaaaaaagatatatcactttcagctcaattattccataattacaagatattaggctagtgggctctttattggttgggtcttatattaatgttatatgctagccttgtaattttgagctcctctatggatgttaattttagtgaaactctatattgaagaaaagatgggactaagggggagacaggaggaggaaagaaagcagagacaggcaaagaatcatagcctgaaattcaacagcaagcatggcttatgaagatcaagttatatttttgcttcatgaatcattgtcagacaaattaagaacatattgtttcttatttatctattgtcaaggattcactatcagacactaagaatgaatcttgattttcataagctctgttgacaccatggagccacagagcataaaacttgcatctaataaagaaagtgcaacatggaacagcagggagtggaataccagcacaactcacagctgcttcctgttcctcgtccctgttttcaggaatgtttcttagcaggaagttttttaatagaccgagaatttgttatatgtattctaagaaaagttgtagttgtagatgcattactctcccaaatcttagagatcagggatgattatgttccatttttgtttggtgagttcccatctttgtatgtacctccttgctcccggctgtcctcctctcctcttccctagtgagtggttaatgagtgttaatgcctaaaccatacttgttttatggacacttctataatggattcgttgcataattttcatgcagtgtatagtgttactagttggaaattcttggaggactcttagctgtctgatgaaattcctagtagaaatttttgttttgaattcctaaagttgaaatatgaaaattatattttaatttgattc

SEQ ID NO: 5 - DNA, 2511 bp, Homo sapiens
agtttttctggtagaaggcggggttctcctcgtacgctgcggagtctctgcggggtgtagaccggaatcctgctgacgggcagagtggatcagggagggagggtcgagacacggtggctgcaggtctgagacaaggctgctccgaggtagtagctctcttgcctggaggtggccattcattcctggagtgctgctgaggagcgagggcccatctggggtctctggaagtcggtgcccaggcctgaaggatagccccccttgcgcttccctgggctgcggccggccttctcagaacgaagggcgtccttccaccccgcggcgcaggtgaccgctgccatggcttttccccatcggccggacgcccctgagctgcctgacttctccatgctgaagaggctggctcgagaccagctcatctatctgctggagcagcttcctggaaaaaaggatttattcattgaggcagatctcatgagccctttggatcgaattgccaatgtctccatcctgaagcaacacgaagtagacaagctatacaaggtggagaacaagccagccctcagctccaatgaacaattgtgcttcttggtcagaccccgcatcaagaatatgcgatacattgccagtcttgtcaatgctgacaaattggctggccgaactcgcaaatacaaagtgatcttcagccctcaaaagttctatgcgtgtgagatggtgcttgaggaagagggaatctatggagatgtgagctgtgatgaatgggccttctctttgctgcctcttgatgtggatctgctgagcatggaactaccagaatttttcagggattactttctggaaggagatcagcgttggatcaacactgtagctcaggccttacaccttctcagcactctctatggaccctttccaaactgctatggaattggcaggtgcgccaagatggcatatgaattgtggaggaacctggaggaggaggaggatggcgaaaccaagggccgaaggccagagattggacatatctttctcttggacagagatgtggactttgtgacagcactttgctcccaagtggtttatgagggcctagtagatgacaccttccgcatcaagtgtgggagtgtcgactttggcccagaagtcacatcctctgacaagagcctgaaggtgctactcaatgccgaggacaaggtgtttaatgagattcggaacgagcacttctccaatgtctttggcttcttgagccagaaggcccggaacttgcaggcccagtatgatcgccggagaggcatggacattaagcagatgaagaatttcgtgtcccaggagctcaagggcctgaaacaggagcaccgcctgctgagtctccatattggggcctgtgaatccatcatgaagaagaaaaccaagcaggatttccaggagctaatcaagactgagcatgcactgctagaggggttcaacatccgggagagcaccagctacattgaggaacacatagaccggcaggtgtcgcctatagaaagcctgcgcctcatgtgccttttgtccatcactgagaatggtttgatccccaaggattaccgatctctgaaaacacagtatctgcagagctatggccctgagcacctgctaaccttctccaatctgcgaagagctgggctcctaacggagcaggcccccggggacaccctcacagccgtggagagtaaagtgagcaagctggtgaccgacaaggctgcaggaaagattactgatgccttcagttctctggccaagaggagcaattttcgtgccatcagcaaaaagctgaatttgatcccacgtgtggacggcgagtatgatctgaaagtgccccgagacatggcttacgtcttcagtggtgcttatgtgcccctgagctgccgaatcattgagcaggtgctagagcggcgaagctggcagggccttgatgaggtggtacggctgctcaactgcagtgactttgcattcacagatatgactaaggaagacaaggcttccagtgagtccctgcgcctcatcttggtggtgttcttgggtggttgtacattctctgagatctcagccctccggttcctgggcagagagaaaggctacaggttcattttcctgacgacagcagtcacaaacagcgctcgccttatggaggccatgagtgaggtgaaagcctgatgtttttcccggccagtgttgacatcttccctgaacacattcctcagtgagatgcaggcatctggcacccagctgctataaccaagtgtccaccaactacctgctaagagccgggagcatggaacgtgttgggatttagagaacattatctgagaaaagagttcacttcctgctcccaggatatttctcttttctgtttatgaagtacaacccatgctgctaagatgcgagcaggaagaggcatcctttgctaaatcctgtttgaatgtcattgtaaataaagcctctgctctcagatgtaaaaaaaaaaaaaaaaaaaaa

SEQ ID NO: 6 - DNA, 2401 bp, Homo sapiens
ggcacgaggggtcgcgctgccgccgttttatttgaagacatcgtccagttctgaccatggactcgcagccatcggcccttagtttccatcccctctagtgggccttcgggggctctactgacgtccctccttcccttggtaccgggccggggaagtgttctcgggcgcgggaggttccgcatgcccaggcctggccaggggagatgaccgatccgtcgctggggctgacagtccccatggcgccgcctctggccccgctccctccccgggacccaaacggggcgggatccgagtggagaaagcccggggccgtgagcttcgccgacgtggccgtgtacttctcccgggaggagtggggctgcctgcggcccgcgcagagggccctgtaccgggacgtgatgcgggagacctacggccacctgggcgcgctcggtgagagccccacctgcttgcctgggccctgcgcctccacaggccctgccgcgcctctgggagctgcgtgtggagttgggggccccggggccgggcaggcggcctcctcgcagcgtggggtttgcgttcttctcccccaggagtcggaggcagcaagccggcgctcatctcctgggtggaggagaaggccgaactgtgggatccggctgcccaggatccggaggtggcgaagtgtccgacagaagcggacccagcagattccagaaacaaggaagaggaaagacaaagggaagggacgggagccctggagaagcccgaccctgtggccgccgggtctcctgggctgaaggctccccaagccccctttgccgggttggagcagctgtccaaggcccggcgccggagtcgcccccgcttttttgcccacccccctgtcccccgagctgaccagcgtcacggctgctacgtgtgcgggaagagcttcgcctggcgctccacactggtggagcacatttacagccacaggggcgagaagcccttccactgcgcagactgcggcaagggcttcggccacgcttcctccctgagcaaacaccgggccatccatcgtggggagcggccccaccgctgtcccgagtgtggtcgggccttcatgcgccgcacggcgctgacttctcacctgcgcgttcacactggcgagaagccctaccgctgcccgcagtgtggccgctgcttcggcctgaagaccggcatggccaagcaccaatgggtccatcggcccgggggcgaggggcgtaggggccggcgccctggggggctgtctgtgaccctgactcctgtccgcggggacctggacccgcctgtgggcttccagctgtatccagagatattccaggaatgtgggtgacggcctaaaaagtgaccatctagacattgtgggcggcccgagatgggctcaggggcccgaacctctgcagcggcctgcagggaggtcccagaatccaccgcaagagctggcctggggtgcggacagtctgatcttgggctctcagcagcctcttctgccagcaccttgctccccgctgccctgggctctccaaggccccctttgctgaggcagggctgaggtgagaaccccccagacctccatacagggaagcaaaagctgtttctcctcccagagatgctaagaggattgaggtagagaagaaccttgttttctctgttgtctttttctttttacttttttaattttttgagacggagttttgctcttgttgcccaggctggagtgcaatggtgcgatctcgactcactgcaacttccacctcctggagtcaagcgattctcctgcctcagccacccaagtagctggaattacaggcacctgccactatgcccggctaactttttgtatttttagtagagatggggtttcaccatgttggctaggctggtctcgaactcctgccctcaggtgatccacccacctctgcctcccaaagtgctgggattacaggcgtgagccacctcacctggccttttcttttttattctttgaccttcccacaagacaatacccattgtctgttttttttgtttatttatttacttattaagacagcatcttgctcctcacccaggctggaatgcagtggtgtgaactgggctcactgcagcctagacctgctgggctcaaggaatcctcctgccccagcctctcagatggctgtgactacaggtgggcaacactatgcctggttaatttttaaatttttttgcagagatggggttcccactatgttgatcaggctggtctcaaactcctcggttcaagcaattcgcccaccttggcctcccaaagtgctgggattacaggggagccactgcactggccttcattgtctttttgctgcacaacctaaaaaaccagtgaccctgtattggaaaaaaaaaaaaaaaaaaaaaa

SEQ ID NO: 7 - DNA, 2384 bp, Homo sapiens
gccatggccgccggccccgcgccgccccccggccgcccccgggcgcagatgccgcatctgaggaaggtgcgaggcggatggagcgggtggtcgtgagcatgcaggaccccgaccagggcgtgaagatgcggagccagcgcctgctggtcaccgtcattccccacgcggtgacaggcagcgacgtcgtgcagtggttggcccagaagttctgcgtctcggaggaggaggccctgcacctgggcgccgtcctggtgcagcatggctacatctacccgctgcgcgacccccgtagcctcatgctccggccagacgagacgccctacaggttccagaccccgtacttctggacaagtaccctgaggccggctgcagagctggactatgccatctacctggccaagaagaacatccgaaaacgggggaccctggtggattatgagaaggactgctatgaccggctacacaagaagatcaaccacgcatgggacctggtgctgatgcaggcgagggagcagctgagggcagccaagcagcgcagcaagggggacaggctggtcattgcgtgccaggagcagacctactggctggtgaacaggcccccgcccggggcccccgatgtgctggagcagggtccagggcggggatcctgcgctgccagccgtgtgctcatgaccaagagtgcagatttccataagcgggagatcgagtacttcaggaaagcgctgggcaggacccgagtgaagtcctccgtctgccttgaggcgtacctgagtttctgcggccagcgtggaccccacgatcccctcgtgtcggggtgcctgcccagcaatccctggatctcagacaatgacgcctactgggtcatgaatgcccccacggtggctgcccccacgaagctccgtgtggagagatggggcttcagcttccgggagctcctggaggaccccgtggggcgggcccacttcatggactttctgggaaaggagttcagtggagaaaacctcagcttctgggaggcatgtgaggagcttcgatatggagcgcaggcccaggtccccaccctggtggatgccgtgtacgagcagttcctggcccccggagctgcccactgggtcaacatcgacagccggaccatggagcagaccctggaggggctgcgccagccccaccgctatgtcctggatgacgcccagctgcacatatacatgctcatgaagaaggactcctacccaaggttcctgaagtctgacatgtacaaggccctcctggcagaggctgggatcccgctggagatgaagagacgcgtgttcccgtttacgtggaggccacggcactcgagccccagccctgcactccttcccacccctgtggagcccacagcggcttgtggccctgggggtggagatggggtggcctagtggacctggcccatctgccactctagtccctgcagctcaacgtcctgcgtgaatgcagcagccacccccgtcttggcccaggtcctgggggctgctgaacccagcaccagtgtccccttgtgcccagggggcccagtcttctgtggggtgcacagcctccctccctccagcaagccctccctgcccagaaggaatgggtccaggtgtggattcccagggagggggttcattggctcagcttgggtcagggcagagcctgttacctgaagagaggtgagaccaaggccacagggagctccaccttctctggtcttcagtccagcactgggtgcccatccccatctctaaaaccagtaaatcagccagcgaatacccggaagcaagatgcacaggcgggcggcttcccacacacccgtcacaagacgcggacatgcaggtctcggcgcgagctctgccccgtccaagagcctctccgctgtcgcccagtgtgagcctggaagaggacccaagagagtgccgtgctgaggctgcctcgaggtcactgccttccggagctgcgcctattcctccctcgccaaacgcgttccagaatttgtccacaggtgcgccggcacctgctttcccacctcgaggccgcggcctcccccccgatttatagacaactctgacattgtcaccccactgacgaggcccgattccatagggtggatccttgccaggcgtccctgatcctccctgcccaagtcttccttcgtgagctggccttgctccccatcccccaagtgcctcaccagtcccccagactgggtgaaggtacagctggctcctttcgggggtgcagcttcaactctctcggcggtagggcggtgccatccccacccatagggctggctcacatccagtcactcccaacagcgtccagcacacaaataaaagacccttgggccctggctctgagaaaaaaaa

SEQ ID NO: 8 - DNA, 1500 bp, Homo sapiens
agactgccgagcagccttgagccgttgagcagctgaacagaggccatgccggggcactccgaggcctgagacgaccacgcctgtgccgctgaggaccttcatcagggctccgtccacttggcccgcttggctgtccaatcacactccagtgtcaaccactggcacccagcagccaagagaggtgtggcgtggccctggggacgcatggctgaggcaggaacaggtgagccgtcccccagcgtggagggcgaacacgggacggagtatgacacgctgccttccgacacagtctccctcagtgactcggactctgacctcagcttgcccggtggtgctgaagtggaagcactgtccccgatggggctgcctggggaggaggattcaggtcctgatgagccgccctcacccccgtcaggcctcctcccagccacggtgcagccattccatctgagaggcatgagctccaccttctcccagcgcagccgtgacatctttgactgcctggagggggcggccagacgggctccatcctctgtggcccacaccagcatgagtgacaacggaggcttcaagcggcccctagcgccctcaggccggtctccagtggaaggcctgggcagggcccatcggagccctgcctcaccaagggtgcctccggtccccgactacgtggcacaccccgagcgctggaccaagtacagcctggaagatgtgaccgaggtcagcgagcagagcaatcaggccaccgccctggccttcctgggctcccagagcctggctgcccccactgactgcgtgtcctccttcaaccaggatccctccagctgtggggaggggagggtcatcttcaccaaaccagtccgaggggtcgaagccagacacgagaggaagagggtcctggggaaggtgggagagccaggcaggggcggccttgggaatcctgccacagacaggggcgagggccctgtggagctggcccatctggccgggcccgggagcccagaggctgaggagtggggcagcccccatggaggcctgcaggaggtggaggcactgtcagggtctgtccacagtgggtctgtgccaggtctcccgccggtggaaactgttggcttccatggcagcaggaagcggagtcgagaccacttccggaacaagagcagcagccccgaggacccaggtgctgaggtctgagagggagatggcccagcctgaccccactggccactgccatcctgctgccttcccagtggggctggtcagggggcagcctggccactgcctagctggaatgggaggaagcctgcaggtggcaccggtggccctggctgcagttctgggcagcatcctcccaagcagagaccttgctgaagctcctggggtgtggggtgtgggctggaagcactggctccctggtagggacaataaaggttttgggtctttcaaaaaaaaaaaaaaaaaaaaaaaaaaaaaaaaaaaaaaaaaaaaaaaaaaaaaaac

SEQ ID NO: 9 - DNA, 60 bp, Homo sapiens
tcatcttcaccaaaccagtccgaggggtcgaagccagacacgagaggaagagggtcctgg

SEQ ID NO: 10 - DNA, 60 bp, Homo sapiens
ctctgctcctgctcctgcctgcatgttctctctgttgttggagcctggagccttgctctc

SEQ ID NO: 11 - DNA, 60 bp, Homo sapiens
tgctcccggctgtcctcctctcctcttccctagtgagtggttaatgagtgttaatgccta

SEQ ID NO: 12 - DNA, 60 bp, Homo sapiens
ccccatctctaaaaccagtaaatcagccagcgaatacccggaagcaagatgcacaggcgg

SEQ ID NO: 13 - DNA, 60 bp, Homo sapiens
ccagaaacaaggaagaggaaagacaaagggaagggacgggagccctggagaagcccgacc

SEQ ID NO: 14 - DNA, 60 bp, Homo sapiens
aagtacaacccatgctgctaagatgcgagcaggaagaggcatcctttgctaaatcctgtt

SEQ ID NO: 15 - DNA, 60 bp, Homo sapiens
acctcacccctgcccggcccaagctctacttgtgtacagtgtatattgtataatagacaa

SEQ ID NO: 16 - DNA, 60 bp, Homo sapiens
ttcccttaattcctcctcccgaccttttttacccccccagttgcagtatttaactgggct

(19) United States
(12) Patent Application Publication
(10) Pub. No.: US 2006/0286579 A1
(43) Pub. Date: Dec. 21, 2006

(54) NORMALIZATION GENES

(75) Inventors: Mark G. Erlander, Encinitas, CA (US); Xiao-Jun Ma, San Diego, CA (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: AviaraDx, Inc., Carlsbad, CA (US)

(21) Appl. No.: 11/422,045

(22) Filed: Jun. 2, 2006

Related U.S. Application Data
(60) Provisional application No. 60/686,989, filed on Jun. 3, 2005.

(51) Int. Cl. C12Q 1/68; C12P 19/34; C12M 1/34

(52) U.S. Cl. 435/6; 435/91.2; 435/287.2

(57) ABSTRACT

This invention provides gene sequence identities and methods for the determination of cellular gene expression and the comparison of expression levels between different gene sequences and between different cells. The gene sequences may be used as reference, normalization, or “housekeeping” gene sequences, the expression of which remains consistent in individual cells, even under different conditions, as well as among cells from different samples and origins.

### RELATED APPLICATIONS

This application claims benefit of priority to U.S. Provisional Patent Application 60/686,989, filed Jun. 3, 2005, which is hereby incorporated by reference as if fully set forth.

### FIELD OF THE INVENTION

This invention relates to the use of cellular gene expression and the comparison of expression levels between different gene sequences and between different cells. The invention provides the identity and use of reference, or normalization, gene sequences, the expression of which remains consistent in individual cells, even under different conditions, as well as among cells from different samples and origins. Methods for the use of these gene sequences as a reference for the comparison of gene expression levels of other gene sequences is provided.

### SUMMARY OF THE INVENTION

This invention relates to the use of gene expression measurements, such as to classify or identify tumors in cell containing samples obtained from a subject in a clinical setting, such as in cases of formalin fixed, paraffin embedded (FFPE) samples as well as fresh samples, that have undergone none to little or minimal treatment (such as simply storage at a reduced, non-freezing, temperature), and frozen samples. The use of gene expression measurements relies upon the ability to detect or measure differential expression of gene sequences in cells. The gene sequences used are those which are differentially expressed in a manner that is relevant to the biological phenotype of interest. The expression level(s) of one or more differentially expressed gene sequences is determined in the cell(s) of a sample or subject, after which the expression level(s) of the one or more sequences are used directly or, in the case of expression of multiple gene sequences, in comparison to each other. One non-limiting example of the latter case is where the expression levels of two gene sequences are determined and then used as a ratio of one to the other.

The comparison of gene expression levels in a cell, as well as between cells of different samples and/or origins and across experiments, is improved when the levels are normalized to a reference. This can be viewed as normalization to a single scale. The use of normalization, such as, but not limited to, where arrays with multiple probes are used to determine gene expression levels, allows for direct array-to-array comparisons. The present invention is related to atandard normalization methods in that the identities of, and methods of using, “housekeeping genes” are provided. While some “housekeeping genes”, like transferrin receptor, actin, and glyceraldehyde-3-phosphate dehydrogenase have been described as being differentially expressed in some situations, the invention is based in part upon the unexpected discovery of gene sequences that are not differentially expressed to appreciable levels in a wide array of cells from different tissue types and displaying different cellular phenotypes.

The reference genes of the invention have been observed to be expressed at consistent levels among cells of at least 39 tumor types. This permits their advantageous use in various settings, such as in a clinical assay to determine expression levels of gene sequences in cells of one of the tumor types. One example of such an assay is the classification of a cell of a sample as being a tumor cell of one tissue as opposed to another.

Thus in a first aspect, the invention provides the identities of eight (8) gene sequences which are expressed at consistent levels in cells of individual tumor types or across two or more of at least 39 tumor types. The sequences may also be expressed at consistent levels in cells of the corresponding normal tissue to each tumor type. The expression of one or more of the 8 reference, or normalization, gene sequences may be embodied in nucleic acid expression, protein expression, or other expression formats, and then compared to the expression levels of gene sequences of interest (hereafter “assay” or “test” gene sequences) in the same or different format. This allows for the normalization of the expression of the assay or test gene sequences relative to one or more of the reference gene sequences of the invention. The expression level(s) of one or more of the 8 reference genes optionally may also be compared between different experiments such that the results of different experiments, such as different arrays, may be compared. The invention provides the advantages of a more accurate determination of gene expression levels in cells.

The details of one or more embodiments of the invention are set forth in the accompanying drawing and the description below. Other features and advantages of the invention will be apparent from the drawing and detailed description, and from the claims.

### DEFINITIONS

As used herein, a “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature. It is appreciated that more than one polynucleotide may be capable of encoding a discrete product. It is also appreciated that alleles and polymorphisms of the human gene sequences may exist and may be used in the practice of the invention to identify the expression level(s) of the gene sequences or an allele or polymorphism thereof. Identification of an allele or polymorphism depends in part upon chromosomal location and ability to recombine during mitosis.

The terms “correlate” or “correlation” or equivalents thereof refer to an association between expression of one or more genes and another event, such as, but not limited to, physiological phenotype or characteristic, such as tumor type.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence. Methods for amplifying mRNA are generally known in the art, and include reverse transcription PCR (RT-PCR) and quantitative PCR (or Q-PCR) or real time PCR. Alternatively, RNA may be directly labeled as the corresponding cDNA by methods known in the art.

By “corresponding”, it is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17).

A “microarray” is a linear or two-dimensional or three dimensional (and solid phase) array of discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, such as of at least about 50/cm², at least about 100/cm², or at least about 500/cm², up to about 1,000/cm² or higher. The arrays may contain less than about 500, about 1000, about 1500, about 2000, about 2500, or about 3000 immobilized polynucleotides in total. As used herein, a DNA microarray is an array of oligonucleotide or polynucleotide probes placed on a chip or other surfaces used to hybridize to amplified or cloned polynucleotides from a sample. Since the position of each particular group of probes in the array is known, the identities of a sample polynucleotides can be determined based on their binding to a particular position in the microarray. As an alternative to the use of a microarray, an array of any size may be used in the practice of the invention, including an arrangement of one or more position of a two-dimensional or three dimensional arrangement in a solid phase to detect expression of a single gene sequence.

Because the invention relies upon the identification of gene expression, some embodiments of the invention determine expression by hybridization of mRNA, or an amplified or cloned version thereof, of a sample cell to a polynucleotide that is unique to a particular gene sequence. Polynucleotides of this type contain at least about 16, at least about 18, at least about 20, at least about 22, at least about 24, at least about 26, at least about 28, at least about 30, or at least about 32 consecutive basepairs of a gene sequence that is not found in other gene sequences. The term “about” as used in the previous sentence refers to an increase or decrease of 1 from the stated numerical value. Other embodiments are polynucleotides of at least or about 50, at least or about 100, at least about or 150, at least or about 200, at least or about 250, at least or about 300, at least or about 350, at least or about 400, at least or about 450, or at least or about 500 consecutive bases of a sequence that is not found in other gene sequences. The term “about” as used in the preceding sentence refers to an increase or decrease of 10% from the stated numerical value. Longer polynucleotides may of course contain minor mismatches (e.g. via the presence of mutations) which do not affect hybridization to the nucleic acids of a sample. Such polynucleotides may also be referred to as polynucleotide probes that are capable of hybridizing to sequences of the genes, or unique portions thereof, described herein. Such polynucleotides may be labeled to assist in their detection. The sequences may be those of mRNA encoded by the genes, the corresponding cDNA to such mRNAs, and/or amplified versions of such sequences. In some embodiments of the invention, the polynucleotide probes are immobilized on an array, other solid support devices, or in individual spots that localize the probes.

In other embodiments of the invention, all or part of a gene sequence may be amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to portions of a gene sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization. Additional methods to detect the expression of expressed nucleic acids include RNAse protection assays, including liquid phase hybridizations, and in situ hybridization of cells.

Alternatively, and in further embodiments of the invention, gene expression may be determined by analysis of expressed protein in a cell sample of interest by use of one or more antibodies specific for one or more epitopes of individual gene products (proteins), or proteolytic fragments thereof, in said cell sample or in a bodily fluid of a subject. The cell sample may be one of breast cancer epithelial cells enriched from the blood of a subject, such as by use of labeled antibodies against cell surface markers followed by fluorescence activated cell sorting (FACS). Such antibodies may be labeled to permit their detection after binding to the gene product. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR.

The terms “label” or “labeled” refer to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of gene expression, mutations in non-coding regions of genes as disclosed herein may also be assayed in the practice of the invention.

“Detection” or “detecting” includes any means of detecting, including direct and indirect determination of the level of gene expression and changes therein.

Unless defined otherwise all technical and scientific terms used herein have the same meaning as commonly understood to one of ordinary skill in the art to which this invention belongs.

### DETAILED DESCRIPTION OF MODES OF PRACTICING THE INVENTION

This invention provides the identities of gene sequences that may be used as normalization, reference, or “housekeeping” sequences in a plurality of cell types and tumor cell types. The expression level(s) of these gene sequences may be advantageously used in methods based on determination of gene expression information to classify tumors.

Thus in a first aspect, the invention provides for the use of 8 particular reference gene sequences that were identified for use in gene expression measurements from at least 39 tumor types. mRNA sequences corresponding to the 8 reference sequences are provided in Example 2 (Sequence Listing) below along with additional identifying information. The listing of the identifying information, including accession numbers and other information, is provided by the following.

| | |
|---|---|
| >Hs.77031_mRNA_1 gi\|16741772\|gb\| | (SEQ ID NO:1) |
| BC016680.1\|BC016680 Homo sapiens | |
| clone MGC:21349 IMAGE:4338754 | |
| polyA = 3 | |
| >Hs.77541_mRNA_1 gi\|12804364\|gb\| | (SEQ ID NO:2) |
| BC003043.1\|BC003043 Homo sapiens | |
| clone MGC:4370 IMAGE:2822973 | |
| polyA = 3 | |
| >Hs.7001_mRNA_1\|gi\|6808256\|emb\| | (SEQ ID NO:3) |
| AL137727.1\|HSM802274 Homo sapiens | |
| mRNA; cDNA DKFZp434M0519 (from clone | |
| DKFZp434M0519); partial cds | |
| polyA = 3 | |
| >Hs.302144_mRNA_1 gi\|11493400\|gb\| | (SEQ ID NO:4) |
| AF130047.1\|AF130047 Homo sapiens | |
| clone FLB3020 polyA = 0 | |
| >Hs.26510_mRNA_2 gi\|11345385\|gb\| | (SEQ ID NO:5) |
| AF308803.1\|AF308803 Homo sapiens | |
| chromosome 15 map 15q26 | |
| polyA = 3 | |
| >Hs.324709_mRNA_2 gi\|12655026\|gb\| | (SEQ ID NO:6) |
| BC001361.1\|BC001361 Homo sapiens | |
| clone MGC:2474 IMAGE:3050694 | |
| polyA = 2 | |
| >Hs.65756_mRNA_3 gi\|3641494\|gb\| | (SEQ ID NO:7) |
| AF035154.1\|AF035154 Homo sapiens | |
| chromosome 16 map 16p13.3 | |
| polyA = 3 | |
| >Hs.165743_mRNA_2 gi\|13543889\|gb\| | (SEQ ID NO:8) |
| BC006O91.1\|BC006091 Homo sapiens | |
| clone MGC:12673 IMAGE:3677524 | |
| polyA = 3 | |

Detection of expression of any of the above reference sequences may be by the same or different methodology as for the other gene sequences described herein. In some embodiments of the invention, the expression levels of gene sequences is measured by detection of expressed sequences in a cell containing sample as hybridizing to the following oligonucleotides, which correspond to the above sequences as indicated by the accession numbers provided.

| | |
|---|---|
| >BC006091 | |
| TCATCTTCACCAAACCAGTCCGAGGGGTCGAAGCCA | (SEQ ID NO:9) |
| GACACGAGAGGAAGAGGGTCCTGG | |
| >BC003043 | |
| CTCTGCTCCTGCTCCTGCCTGCATGTTCTCTCTGTT | (SEQ ID NO:10) |
| GTTGGAGCCTGGAGCCTTGCTCTC | |
| >AF130047 | |
| TGCTCCCGGCTGTCCTCCTCTCCTCTTCCCTAGTGA | (SEQ ID NO:11) |
| GTGGTTAATGAGTGTTAATGCCTA | |
| >AF035154 | |
| CCCCATCTCTAAAACCAGTAAATCAGCCAGCGAATA | (SEQ ID NO:12) |
| CCCGGAAGCAAGATGCACAGGCGG | |
| >BC001361 | |
| CCAGAAACAAGGAAGAGGAAAGACAAAGGGAAGGGA | (SEQ ID NO:13) |
| CGGGAGCCCTGGAGAAGCCCGACC | |
| >AF308803 | |
| AAGTACAACCCATGCTGCTAAGATGCGAGCAGGAAG | (SEQ ID NO:14) |
| AGGCATCCTTTGCTAAATCCTGTT | |
| >BC016680 | |
| ACCTCACCCCTGCCCGGCCCAAGCTCTACTTGTGTA | (SEQ ID NO:15) |
| CAGTGTATATTGTATAATAGACAA | |
| >AL137727 | |
| TTCCCTTAATTCCTCCTCCCGACCTTTTTTACCCCC | (SEQ ID NO:16) |
| CCAGTTGCAGTATTTAACTGGGCT | |

In some embodiments, the invention provides a method of determining the expression level of one or more of the above reference gene sequences in a cell, such as in one or more cells of a cell containing sample from a subject. The method comprises determining the expression level(s) of one or more of the reference gene sequences which comprises, or hybridizes to, at least 20 nucleotides of one or more of SEQ ID NOs: 1-16. Thus the invention provides for the determination of the expression levels of a reference gene sequence by measuring all or part of the expressed sequence corresponding thereto.

In other embodiments, the invention provides a method of determining the expression level of one or more gene sequences of interest in a cell of a sample, such as one or more cells of a sample from a subject. The method comprises determining the expression level(s) of said one or more gene sequences in said cell, and comparing said expression level(s) to the expression level of a reference gene sequence as described above. The expression level(s) of one or more reference gene sequences of the invention provides a means to “normalize” the expression data from gene sequences of interest for comparison of data from a sample. Stated differently, expression of a gene sequence of interest is calculated in a manner “relative to” expression of one or more reference gene sequence of the invention. The normalization may also be used for comparisons between samples, especially when they are conducted in separate experiments. Alternatively, the reference gene sequences may be used in the same manner as other “housekeeping” gene sequences known to the skilled person.

The methods of the invention may be advantageously used in an array based format, and thus a plurality of gene sequences may be evaluated for their expression at the same time. One or more of the reference gene sequences of the invention may also be evaluated as part of the same experiment. As a non-limiting example, the use of an array based format may include reverse transcription of mRNA corresponding to a reference gene sequence to produce cDNA, which is then detected.

Alternatively, the expression level(s) are determined by means comprising amplification of all or part of the reference gene sequences described above. One non-limiting means of amplification uses quantitative PCR, including the use of a multiplex format to determine expression of a reference gene sequence as well as a gene sequence of interest (assay or test sequence). Another non-limiting means uses RNA amplification, and . may be based upon labeling of the resultant RNA molecules for subsequent detection.

In some amplification based embodiments of the invention, the amplified sequence may be within 300 nucleotides of the polyadenylation sites of the transcripts. In embodiments comprising quantitative PCR, amplification may be of at least about 50 to about 80 nucleotides of the transcripts.

In some non-limiting embodiments, the reference gene sequences of the invention are used in a method of classifying a cell containing sample as including a tumor cell of (or from) a type of tissue or a tissue origin. The classifying is based upon a comparison of the expression levels of a plurality of transcribed assay sequences in the cells of the sample to their expression levels in known tumor samples and/or known non-tumor samples. As used herein, “a plurality” refers to the state of two or more. Such use in classification will be used in additional description of the invention below as a non-limiting example.

The reference gene sequences of the invention may also be used in the classification of a sample as containing cells from a tissue or organ site, without limitation to tumor cells. In some embodiments of the invention, a sample is classified as being from one of the following 24 tissue or organ origins: Adrenal, Bladder, Bone, Brain, Breast, Cervix, Endometrium, Esophagus, Gall Bladder, Kidney, Larynx, Liver, Lung, Lymph Node, Ovary, Pancreas, Prostate, Skin, Soft Tissue, Small/Large Bowel, Stomach, Testes, Thyroid, and Uterus.

In additional non-limiting embodiments, the invention is described with respect to human subjects. However, samples from other subjects may also be used. All that is necessary is the ability to assess the expression levels of gene sequences in a plurality of known samples such that the expression levels in an unknown or test sample may be compared. Thus the invention may be applied to samples from any organism for which a plurality of expressed sequences, and a plurality of known samples, are available. One non-limiting example is application of the invention to mouse samples, based upon the availability of the mouse genome to permit detection of expressed murine sequences and the availability of known mouse tumor samples or the ability to obtain known samples. Thus, the invention is contemplated for use with other samples, including those of mammals, primates, and animals used in clinical testing (such as rats, mice, rabbits, dogs, cats, and chimpanzees) as non-limiting examples.

The invention provides for the normalization of the expression levels of the assay gene sequences with one or more of the reference gene sequences disclosed herein. Thus, the classifying may alternatively be based upon a comparison of the expression levels of the assay sequences to the expression of reference sequences in the same samples, relative to, or based on, the same comparison in known tumor samples and/or known non-tumor samples. As a non-limiting example, the normalized expression levels of the assay gene sequences may be determined in a set of known tumor samples to provide a database against which the normalized expression levels detected or determined in a cell containing sample from a subject is compared.

The expression level(s) of assay sequence(s) in a sample also may be compared to the expression level(s) of said sequence(s) in normal or non-cancerous cells, preferably from the same sample or subject. In other embodiments of the invention utilizing Q-PCR or real time Q-PCR, the expression levels may be compared to expression levels of reference gene sequences in the same sample or a ratio of expression levels may be used.

The invention is readily practiced with the use of cell containing samples, although any nucleic acid containing sample which may be assayed for gene expression levels may be used in the practice of the invention. Without limiting the invention, a sample of the invention may be one that is suspected or known to contain tumor cells. Alternatively, a sample of the invention may be a “tumor sample” or “tumor containing sample” or “tumor cell containing sample” of tissue or fluid isolated from an individual suspected of being afflicted with, or at risk of developing, cancer. Non-limiting examples of samples for use with the invention include a clinical sample, such as, but not limited to, a fixed sample, a fresh sample, or a frozen sample. The sample may be an aspirate, a cytological sample (including blood or other bodily fluid), or a tissue specimen, which includes at least some information regarding the in situ context of cells in the specimen, so long as appropriate cells or nucleic acids are available for determination of gene expression levels.

Non-limiting examples of fixed samples include those that are fixed with formalin or formaldehyde (including FFPE samples), with Boudin's, glutaldehyde, acetone, alcohols, or any other fixative, such as those used to fix cell or tissue samples for immunohistochemistry (IHC). Other examples include fixatives that precipitate cell associated nucleic acids and proteins. Given possible complications in handling frozen tissue specimens, such as the need to maintain its frozen state, the invention may be practiced with non-frozen samples, such as fixed samples, fresh samples, including cells from blood or other bodily fluid or tissue, and minimally treated samples.

In some embodiments of the invention, the sample is classified as containing a tumor cell of a type selected from the following 39: adrenal gland, brain, breast, carcinoid-intestine, cervix-adenocarcinoma, cervix-squamous, endometrium, gall bladder, germ cell-ovary, GIST, kidney, leiomyosarcoma, liver, lung-adenocarcinoma-large cell, lung-small cell, lung-squamous, lymphoma-B cell, lymphoma-Hodgkin's, lymphoma-T cell, meningioma, mesothelioma, osteosarcoma, ovary-clear cell, ovary-serous, pancreas, prostate, skin-basal cell, skin-melanoma, skin-squamous, small and large bowel, soft tissue-liposarcoma, soft tissue-MFH, soft tissue-sarcoma-synovial, stomach-adenocarcinoma, testis-other (or non-seminoma), testis-seminoma, thyroid-follicular-papillary, thyroid-medullary, and urinary bladder.

The reference gene sequences of the invention may also be used in the classification of a tumor cell of a type selected from the following 53: Adenocarcinoma of Breast, Adenocarcinoma of Cervix, Adenocarcinoma of Esophagus, Adenocarcinoma of Gall Bladder, Adenocarcinoma of Lung, Adenocarcinoma of Pancreas, Adenocarcinoma of Small-Large Bowel, Adenocarcinoma of Stomach, Astrocytoma, Basal Cell Carcinoma of Skin, Cholangiocarcinoma of Liver, Clear Cell Adenocarcinoma of Ovary, Diffuse Large B-Cell Lymphoma, Embryonal Carcinoma of Testes, Endometrioid Carcinoma of Uterus, Ewings Sarcoma, Follicular Carcinoma of Thyroid, Gastrointestinal Stromal Tumor, Germ Cell Tumor of Ovary, Germ Cell Tumor of Testes, Glioblastoma Multiforme, Hepatocellular Carcinoma of Liver, Hodgkin's Lymphoma, Large Cell Carcinoma of Lung, Leiomyosarcoma, Liposarcoma, Lobular Carcinoma of Breast, Malignant Fibrous Histiocytoma, Medulary Carcinoma of Thyroid, Melanoma, Meningioma, Mesothelioma of Lung, Mucinous Adenocarcinoma of Ovary, Myofibrosarcoma, Neuroendocrine Tumor of Bowel, Oligodendroglioma, Osteosarcoma, Papillary Carcinoma of Thyroid, Pheochromocytoma, Renal Cell Carcinoma of Kidney, Rhabdomyosarcoma, Seminoma of Testes, Serous Adenocarcinoma of Ovary, Small Cell Carcinoma of Lung, Squamous Cell Carcinoma of Cervix, Squamous Cell Carcinoma of Esophagus, Squamous Cell Carcinoma of Larynx, Squamous Cell Carcinoma of Lung, Squamous Cell Carcinoma of Skin, Synovial Sarcoma, T-Cell Lymphoma, and Transitional Cell Carcinoma of Bladder.

The methods of the invention may also be applied to classify a cell containing sample as containing a tumor cell of a tumor of a subset of any of the above sets. The size of the subset will usually be small, composed of two, three, four, five, six, seven, eight, nine, or ten of the tumor types described above. Alternatively, the size of the subset may be any integral number up to the full size of the set. Thus embodiments of the invention include classification among 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 of the above types. In some embodiments, the subset will be composed of tumor types that are of the same tissue or organ type. Alternatively, the subset will be composed of tumor types of different tissues or organs. In some embodiments, the subset will include one or more types selected from adrenal gland, brain, carcinoid-intestine, cervix-adenocarcinoma, cervix-squamous, gall bladder, germ cell-ovary, GIST, leiomyosarcoma, liver, meningioma, osteosarcoma, skin-basal cell, skin-squamous, soft tissue-liposarcoma, soft tissue-MFH, soft tissue-sarcoma-synovial, testis-other (or non-seminoma), testis-seminoma, thyroid-follicular-papillary, and thyroid-medullary.

In other embodiments, the gene expression levels of other gene sequences may be determined along with the above described determinations of expression levels for use in classification. One non-limiting example of this is seen in the case of a microarray based platform to determine gene expression, where the expression of other gene sequences is also measured. Where those other expression levels are not used in classification, they may be considered the results of “excess” transcribed sequences and not critical to the practice of the invention. In some embodiments, the invention includes the use of expression level(s) from one or more “excess” gene sequences, such as those which may provide information redundant to one or more other gene sequences used in a classification method.

As would be understood by the skilled person, detection of expression of any of the reference gene sequences, like the sequences provided in Example 2 (Sequence Listing), may be performed by the detection of expression of any appropriate portion or fragment of these sequences. Preferably, the portions are sufficiently large to contain unique sequences relative to other sequences expressed in a cell containing sample. Moreover, the skilled person would recognize that the disclosed sequences represent one strand of a double stranded molecule and that either strand may be detected as an indicator of expression of the disclosed sequences. This follows because the disclosed sequences are expressed as RNA molecules in cells which are preferably converted to cDNA molecules for ease of manipulation and detection. The resultant cDNA molecules may have the sequences of the expressed RNA as well as those of the complementary strand thereto. Thus either the RNA sequence strand or the complementary strand may be detected. Of course is it also possible to detect the expressed RNA without conversion to cDNA.

As used herein, a “tumor sample” or “tumor containing sample” or “tumor cell containing sample” or variations thereof, refer to cell containing samples of tissue or fluid isolated from an individual suspected of being afflicted with, or at risk of developing, cancer. The samples may contain tumor cells which may be isolated by known methods or other appropriate methods as deemed desirable by the skilled practitioner. These include, but are not limited to, microdissection, laser capture microdissection (LCM), or laser microdissection (LMD) before use in the instant invention. Alternatively, undissected cells within a “section” of tissue may be used. Non-limiting examples of such samples include primary isolates (in contrast to cultured cells) and may be collected by any non-invasive or minimally invasive means, including, but not limited to, ductal lavage, fine needle aspiration, needle biopsy, the devices and methods described in U.S. Pat. No. 6,328,709, or any other suitable means recognized in the art. Alternatively, the sample may be collected by an invasive method, including, but not limited to, surgical biopsy.

The detection and measurement of transcribed sequences may be accomplished by a variety of means known in the art or as deemed appropriate by the skilled practitioner. Essentially, any assay method may be used as long as the assay reflects, quantitatively or qualitatively, expression of the transcribed sequence being detected.

The ability to use the disclosed sequences as reference sequences is provided by the recognition of their consistent expression levels in cells of a variety of biological phenotypes and not by the form of the assay used to determine the actual level of expression. An assay of the invention may utilize any identifying feature of a individual reference gene sequence as disclosed herein as long as the assay reflects, quantitatively or qualitatively, expression of the gene sequence in the “transcriptome” (the transcribed fraction of genes in a genome) or the “proteome” (the translated fraction of expressed genes in a genome). Additional assays include those based on the detection of polypeptide fragments of the relevant member or members of the proteome. Non-limiting examples of the latter include detection of proteolytic fragments found in a biological fluid, such as blood or serum. Identifying features include, but are not limited to, unique nucleic acid sequences used to encode (DNA), or express (RNA), said gene or epitopes specific to, or activities of, a protein encoded by a reference gene sequence.

In some embodiments, all or part of a reference gene sequence is amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to portions of a gene sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing reference gene sequences) of the invention under conditions which allow for their hybridization. Additional methods to detect the expression of expressed nucleic acids include RNAse protection assays, including liquid phase hybridizations, and in situ hybridization of cells.

Alternatively, the expression of reference gene sequences in FFPE samples may be detected as disclosed in U.S. applications 60/504,087, filed Sep. 19, 2003, Ser. No. 10/727,100, filed Dec. 2, 2003, and Ser. No. 10/773,761, filed Feb. 6, 2004 (all three of which are hereby incorporated by reference as if fully set forth). Briefly, the expression of all or part of an expressed gene sequence or transcript may be detected by use of hybridization mediated detection (such as, but not limited to, microarray, bead, or particle based technology) or quantitative PCR mediated detection (such as, but not limited to, real time PCR and reverse transcriptase PCR) as non-limiting examples. The expression of all or part of an expressed polypeptide may be detected by use of immunohistochemistry techniques or other antibody mediated detection (such as, but not limited to, use of labeled antibodies that bind specifically to at least part of the polypeptide relative to other polypeptides) as non-limiting examples. Additional means for analysis of gene expression are available, including detection of expression within an assay for global, or near global, gene expression in a sample (e.g. as part of a gene expression profiling analysis such as on a microarray). Non-limiting examples linear RNA amplification and those described in U.S. patent application Ser. No. 10/062,857 (filed on Oct. 25, 2001), as well as U.S. Provisional Patent Applications 60/298,847 (filed Jun. 15, 2001) and 60/257,801 (filed Dec. 22, 2000), all of which are hereby incorporated by reference in their entireties as if fully set forth.

In embodiments using a nucleic acid based assay to determine expression includes immobilization of one or more reference gene sequences on a solid support, including, but not limited to, a solid substrate as an array or to beads or bead based technology as known in the art. Alternatively, solution based expression assays known in the art may also be used. The immobilized gene sequence(s) may be in the form of polynucleotides that are unique or otherwise specific to the gene(s) such that the polynucleotides would be capable of hybridizing to the DNA or RNA of said gene(s). These polynucleotides may be the full length of the gene(s) or be short sequences of the reference genes (up to one nucleotide shorter than the full length sequence known in the art by deletion from the 5′ or 3′ end of the sequence) that are optionally minimally interrupted (such as by mismatches or inserted non-complementary basepairs) such that hybridization with a DNA or RNA corresponding to the genes is not affected. In some embodiments, the polynucleotides used are from the 3′ end of the gene, such as within about 350, about 300, about 250, about 200, about 150, about 100, or about 50 nucleotides from the polyadenylation signal or polyadenylation site of a gene or expressed sequence. Polynucleotides containing mutations relative to the sequences of the disclosed reference genes may also be used so long as the presence of the mutations still allows hybridization to produce a detectable signal. Thus the practice of the present invention is unaffected by the presence of minor mismatches between the disclosed sequences and those expressed by cells of a subject's sample. A non-limiting example of the existence of such mismatches are seen in cases of sequence polymorphisms between individuals of a species, such as individual human patients within Homo sapiens.

As will be appreciated by those skilled in the art, some reference gene sequences include 3′ poly A (or poly T on the complementary strand) stretches that do not contribute to the uniqueness of the disclosed sequences. The invention may thus be practiced with gene sequences lacking the 3′ poly A (or poly T) stretches. The uniqueness of the disclosed sequences refers to the portions or entireties of the sequences which are found only in nucleic acids, including unique sequences found at the 3′ untranslated portion thereof. Some unique sequences for the practice of the invention are those which contribute to the consensus sequences for the genes such that the unique sequences will be useful in detecting expression in a variety of individuals rather than being specific for a polymorphism present in some individuals. Alternatively, sequences unique to an individual or a subpopulation may be used. The unique sequences may be the lengths of polynucleotides of the invention as described herein.

In additional embodiments of the invention, polynucleotides having sequences present in the 3′ untranslated and/or non-coding regions of reference gene sequences are used to detect expression levels in cell containing samples of the invention. Such polynucleotides may optionally contain sequences found in the 3′ portions of the coding regions of reference gene sequences. Polynucleotides containing a combination of sequences from the coding and 3′ non-coding regions preferably have the sequences arranged contiguously, with no intervening heterologous sequence(s).

Alternatively, the invention may be practiced with polynucleotides having sequences present in the 5′ untranslated and/or non-coding regions of reference gene sequences to detect the level of expression in cells and samples of the invention. Such polynucleotides may optionally contain sequences found in the 5′ portions of the coding regions. Polynucleotides containing a combination of sequences from the coding and 5′ non-coding regions may have the sequences arranged contiguously, with no intervening heterologous sequence(s). The invention may also be practiced with sequences present in the coding regions of gene sequences.

The polynucleotides of some embodiments contain sequences from 3′ or 5′ untranslated and/or non-coding regions of at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, or at least about 46 consecutive nucleotides. The term “about” as used in the previous sentence refers to an increase or decrease of 1 from the stated numerical value. Other embodiments use polynucleotides containing sequences of at least or about 50, at least or about 100, at least about or 150, at least or about 200, at least or about 250, at least or about 300, at least or about 350, or at least or about 400 consecutive nucleotides. The term “about” as used in the preceding sentence refers to an increase or decrease of 10% from the stated numerical value.

Sequences from the 3′ or 5′ end of gene coding regions as found in polynucleotides of the invention are of the same lengths as those described above, except that they would naturally be limited by the length of the coding region. The 3′ end of a coding region may include sequences up to the 3′ half of the coding region. Conversely, the 5′ end of a coding region may include sequences up the 5′ half of the coding region. Of course the above described sequences, or the coding regions and polynucleotides containing portions thereof, may be used in their entireties.

In another embodiment of the invention, polynucleotides containing deletions of nucleotides from the 5′ and/or 3′ end of reference gene sequences may be used. The deletions are preferably of 1-5, 5-10, 10-15, 15-20, 20-25, 25-30, 30-35, 35-40, 40-45, 45-50, 50-60, 60-70, 70-80, 80-90, 90-100, 100-125, 125-150, 150-175, or 175-200 nucleotides from the 5′ and/or 3′ end, although the extent of the deletions would naturally be limited by the length of the sequences and the need to be able to use the polynucleotides for the detection of expression levels.

Other polynucleotides of the invention from the 3′ end of reference gene sequences include those of primers and optional probes for quantitative PCR. Preferably, the primers and probes are those which amplify a region less than about 350, less than about 300, less than about 250, less than about 200, less than about 150, less than about 100, or less than about 50 nucleotides from the from the polyadenylation signal or polyadenylation site of a reference gene or expressed sequence. The size of a PCR amplicon of the invention may be of any size, including at least or about 50, at least or about 100, at least about or 150, at least or about 200, at least or about 250, at least or about 300, at least or about 350, or at least or about 400 consecutive nucleotides, all with inclusion of the portion complementary to the PCR primers used.

Other polynucleotides for use in the practice of the invention include those that have sufficient homology to reference gene sequences to detect their expression by use of hybridization techniques. Such polynucleotides preferably have about or 95%, about or 96%, about or 97%, about or 98%, or about or 99% identity with the reference gene sequences to be used. Identity is determined using the BLAST algorithm, as described above. The other polynucleotides for use in the practice of the invention may also be described on the basis of the ability to hybridize to polynucleotides of the invention under stringent conditions of about 30% v/v to about 50% formamide and from about 0.01M to about 0.15M salt for hybridization and from about 0.01M to about 0.15M salt for wash conditions at about 55 to about 65° C. or higher, or conditions equivalent thereto.

In a further embodiment of the invention, a population of single stranded nucleic acid molecules comprising one or both strands of a human gene sequence is provided as a probe such that at least a portion of said population may be hybridized to one or both strands of a nucleic acid molecule quantitatively amplified from RNA of a cell or sample of the invention. The population may be only the antisense strand of a human gene sequence such that a sense strand of a molecule from, or amplified from, a cell may be hybridized to a portion of said population. The population preferably comprises a sufficiently excess amount of said one or both strands of a human gene sequence in comparison to the amount of expressed (or amplified) nucleic acid molecules containing a complementary gene sequence.

In additional embodiments, the invention may be practiced by analyzing gene expression from single cells or homogenous cell populations which have been dissected away from, or otherwise isolated or purified from, contaminating cells of a sample as present in a simple biopsy. One advantage provided by these embodiments is that contaminating, non-tumor cells (such as infiltrating lymphocytes or other immune system cells) may be removed as so be absent from affecting the genes identified or the subsequent analysis of gene expression levels as provided herein. Such contamination is present where a biopsy is used to generate gene expression profiles.

The invention further provides kits for the determination or measurement of reference gene expression levels in a cell containing sample as described herein. A kit will typically comprise one or more reagents to detect reference gene expression as described herein for the practice of the present invention. Non-limiting examples include polynucleotide probes or primers for the detection of expression levels, one or more enzymes used in the methods of the invention, and one or more tubes for use in the practice of the invention. In some embodiments, the kit will include an array, or solid media capable of being assembled into an array, for the detection of gene expression as described herein. In other embodiments, the kit may comprise one or more antibodies that is immunoreactive with epitopes present on a polypeptide which indicates expression of a reference gene sequence. In some embodiments, the antibody will be an antibody fragment.

A kit of the invention may also include instructional materials disclosing or describing the use of the kit or a primer or probe of the present invention in a method of the invention as provided herein. A kit may also include additional components to facilitate the particular application for which the kit is designed. Thus, for example, a kit may additionally contain means of detecting the label (e.g. enzyme substrates for enzymatic labels, filter sets to detect fluorescent labels, appropriate secondary labels such as a sheep anti-mouse-HRP, or the like). A kit may additionally include buffers and other reagents recognized for use in a method of the invention.

Having now generally described the invention, the same will be more readily understood through reference to the following examples which are provided by way of illustration, and are not intended to be limiting of the present invention, unless specified.

### EXAMPLES

### Example 1

Expression of one or more reference gene sequences of the invention is determined in a cell of a sample in combination with expression of the estrogen receptor (ERα), which has been observed to be expressed in correlation with some breast and ovarian cancers. The expression level of ERα is normalized to the expression of one or more reference gene sequences.

Quantitative PCR used to determine ERα and reference gene expression results in the generation of Cₜ values. The Cₜ value for ERα may be divided by the Cₜ value for a reference gene sequence to result in a normalized Cₜ value. This value may be compared to the normalized (to the same reference gene sequence) value of ERα expression in other cells.

The expression of more than one, up to 8, of the sequences is determined and then averaged to derive a mean Cₜ value, which is then used in comparison to the expression level of ERα to produce a normalized Cₜ value.

EXAMPLE 2
mRNA Sequences (Sequence Listing)

>Hs.77031_mRNA_1 gi|16741772|gb|BC016680.1|
BC016680 Homo sapiens clone MGC:21349 IMAGE:
4338754 polyA = 3
GTGGCGGCGGAGGCGGCGGAGGCCAGGGAGGAAGATGTCGTAATGAGCGA
TCCACAGACCAGCATGGCTGCCACTGCTGCTGTGAGTCCCAGTGACTACC
TGCAGCCTGCCGCCTCCACCACCCAGGACTCCCAGCCATCTCCCTTAGCC
CTGCTTGCTGCAACATGTAGCAAAATTGGCCCTCCAGCAGTTGAAGCTGC
TGTGACACCTCCTGCTCCCCCACAGCCCACACCGCGGAAACTTGTCCCTA
TCAAACCTGCCCCTCTCCCTCTCAGCCCCGGCAAGAATAGCTTTGGAATC
TTGTCCTCCAAAGGAAATATACTTCAGATTCAGGGGTCACAACTGAGCGC
CTCCTATCCTGGAGGGCAGCTGGTGTTCGCTATCCAGAATCCCACCATGA
TCAACAAAGGGACCCGATCAAATGCCAATATCCAGTACCAGGCGGTCCCT
CAGATTCAGGCAAGCAATTCCCAAACCATCCAAGTACAGCCCAATCTCAC
CAACCAGATCCAGATCATCCCTGGCACCAACCAAGCCATCATCACCCCCT
CACCGTCCAGTCACAAGCCTGTCCCCATCAAGCCAGCCCCCATCCAGAAG
TCGAGTACGACCACCACCCCCGTGCAGAGCGGGGCCAATGTGGTGAAGTT
GACAGGTGGGGGCGGCAATGTGACGCTCACTCTGCCCGTCAACAACCTCG
TGAACGCCAGTGACACCGGGGCCCCTACTCAGCTCCTCACTGAAAGCCCC
CCAACCCCGCTGTCTAAGACTAACAAGAAAGCAAGGAAGAAGAGCCTTCC
TGCCTCCCAGCCCCCTGTGGCTGTGGCTGAGCAGGTGGAGACGGTGCTGA
TCGAGACCACCGCGGACAACATCATCCAGGCAGGAAATAACCTGCTCATT
GTTCAGAGCCCTGGTGGGGGCCAGCCAGCTGTGGTCCAGCAGGTCCAGGT
GGTGCCCCCCAAGGCCGAGCAGCAGCAGGTGGTACAGATCCCCCAGCAGG
CTCTGCGGGTGGTGCAGGCGGCATCTGCCACCCTCCCCACTGTACCCCAG
AAGCCCTCCCAGAACTTTCAGATCCAGGCAGCTGAGCCGACACCTACTCA
GGTCTACATCCGCACGCCTTCCGGTGAGGTGCAGACAGTCCCTGCATCCC
GTGCTCCCCATCTGAGTGGGACCAGCAAAAAGCACTCAGCTGCAATTCTC
CGAAAAGAGCGTCCCCTGCCAAAGATTGCCCCAGCCGGGAGCATCATCAG
CCTGAATGCAGCCCAGTTGGCGGCAGCTGCCCAGGCAATGCAGACCATCA
ACATCAATGGTGTCCAGGTCCAGGGCGTGCCTGTCACCATCACCAACACA
GGCGGGCAGCAGCAGCTGACAGTGCAGAATGTTTCTGGGAACAACCTGAC
CATCAGTGGGCTGAGCCCCACCCAGATCCAGCTGCAAATGGAACAAGCCC
TGGCCGGAGAGACCCAGCCCGGGGAGAAGCGGCGCCGCATGGCCTGCACG
TGTCCCAACTGCAAGGATGGGGAGAAGAGGTCTGGAGAGCAGGGCAAGAA
GAAGCACGTGTGCCACATCCCCGACTGTGGCAAGACGTTCCGTAAGACGT
CCTTGCTGCGTGCCCATGTGCGCCTGCACACTGGCGAGCGGCCCTTTGTC
TGCAACTGGTTCTTCTGTGGGAAGAGGTTCTGCGCCCAGTGTCAGAAGCG
CTTCATGAGGAGTGACCACCTCACCAAGCATTACAAGACCCACCTGGTCA
CGAAGAACTTGTAAGGCCAACTGCGGCGGGAGGCCCTGAAGATGCAGTCC
CCCACCTGTGTCCTCCCTGGGCCCCTGGTGGAAAGGAGCCCTGTGGCTGC
CTTGGGCCTGCCCTCAGCCCCACTCCTGTTCTGCAACTGTCCCCACAGGA
AGGGGCTCTGTTCCCTGTATTGTCCTCCTTCTGAAGCCCCTTGGCTCTGC
CTTGGCCCTTCCCCTCACCACGAGCTCCCGGCCTGCCCAGACTGTGGACA
CTGGCCGTGCCCAATGAGACGTTCTAAACCAGGACGCGTGGGAACCCTTA
TTTCCAAAGGAAAAACATGCATTTCACTCCGTCGAGGAGCAAAGTGAGCC
CCTACCCCCCACCCCGATCCCCGCTCCCAACACTGCCGGAGTCGCGTCAT
GCCATGCCCCCTCTCCTGCACCTCCCTGGCCCTGCCGGCCACTGTGGACG
CCCTGGGGCTTGGCACCCACCCAGCACATTCCAGCTCTATTTAAAAAGTA
AAGACACCCACCGACTCCTGATCCCCCTCTTTTTCTATGGAGAACGTTGC
CTTATACTCTCTACTTCAGATGATGAACACTGTGTACTGTGTGTGCTTTA
AAGAAGTTTTATTTAATTGCTCCCTTCTTCCTTTCCTTGTTATTCACCTC
CCTGATGCCTGCTTTCAGTTGAGGGTTGGGGGCAATGATGAGCATATGAA
TTTTTTCTCACTCTAGCAATTCCCTTTTCTAAATGACACAGCATTTAAAC
TCAAATCTGGATTCAGATAACAGCACCTGCACATCCTGCACCTCCTCCCT
CTCCCTTCACCTCACCCCTGCCCGGCCCAAGCTCTACTTGTGTACAGTGT
ATATTGTATAATAGACAATTGTGTCTACTACATGTTTAAAAACACATTGC
TTGTTATTTTTGAGGCTTTTAAATTAAACAAAAATCCAACTTTAAAAAAA
AAAAAAAA
>Hs.77541_mRNA_1 gi|12804364|gb|BC003043.1|
BC003043 Homo sapiens clone MGC:4370
IMAGE:2822973 polyA = 3
CCCGCGTCGGTGCCCGCGCCCCTCCCCGGGCCCCGCCATGGGCCTCACCG
TGTCCGCGCTCTTTTCGCGGATCTTCGGGAAGAAGCAGATGCGGATTCTC
ATGGTTGGCTTGGATGCGGCTGGCAAGACCACAATCCTGTACAAACTGAA
GTTGGGGGAGATTGTCACCACCATCCCAACCATAGGCTTCAATGTAGAAA
CAGTGGAATATAAGAACATCTGTTTCACAGTCTGGGACGTGGGAGGCCAG
GACAAGATTCGGCCTCTGTGGCGGCACTACTTCCAGAACACTCAGGGCCT
CATCTTTGTGGTGGACAGTAATGACCGGGAGCGGGTCCAAGAATCTGCTG
ATGAACTCCAGAAGATGCTGCAGGAGGACGAGCTGCGGGATGCAGTGCTG
CTGGTATTTGCCAACAAGCAGGACATGCCCAACGCCATGCCCGTGAGCGA
GCTGACTGACAAGCTGGGGCTACAGCACTTACGCAGCCGCACGTGGTATG
TCCAGGCCACCTGTGCCACCCAAGGCACAGGTCTGTACGATGGTCTGGAC
TGGCTGTCCCACGAGCTGTCAAAGCGCTAACCAGCCAGGGGCAGGCCCCT
GATGCCCGGAAGCTCCTGCGTGCATCCCCGGATGACCATACTCCCGGACT
CCTCAGGCAGTGCCCTTTCCTCCCACTTTTCCTCCCCCATAGCCACAGGC
CTCTGCTCCTGCTCCTGCCTGCATGTTCTCTCTGTTGTTGGAGCCTGGAG
CCTTGCTCTCTGGGCACAGAGGGGTCCACTCTCCTGCCTGCTGGGACCTA
TGGAAGGGGCTTCCTGGCCAAGGCCCCCTCTTCCAGAGGAGGAGCAGGGA
TCTGGGTTTCCTTTTTTTTTTCTGTTTTGGGTGTACTCTAGGGGCCAGGT
TGGGAGGGGGAAGGTGAGGGCTTCGGGTGGTGCTATAATGTGGCACTGGA
TCTTGAGTAATAAATTTGCTGTGGTTTGAAAAAAAAAAAAAAAAAAAAA
>Hs.7001_mRNA_1 gi|6808256|emb|AL137727.1|
HSM802274 Homo sapiens mRNA; cDNA DKFZp434M0519
(from clone DKFZp434M0519); partial cds polyA = 3
GTGGCGGTGGCTGCGGCGACGGCAGAGGCGAAGGGAGCCGGATCGCCGAC
CTGAGCGGGAGGCGGCGGTGGCGGCCATGGCGGCAGATGGAGAGCGTTCC
CCGCTGCTGTCTGAGCCCATCGACGGTGGCGCGGGCGGCAACGGTTTAGT
GGGGCCCGGCGGGAGTGGGGCTGGGCCCGGGGGAGGCCTGACCCCCTCCG
CACCACCGTACGGAGCCGGTAAACATGCCCCGCCCCAGGGTAAGCCGGGG
CGGGTCCGAGGTGCTCCCCGGGGTACTCTGAAAGCCGGGGAGGGGGCGGG
ACCGAGGGCGGAGGCGGGTCCCAGTCGCCAGGTGCGGGACTGCTGCACCT
GTGACTGGGCGAGGCTTCCTTCCCTCCGTAATCGCGACCACAGCCTAGGG
ACGGAAGGGGGTTCTGAGCAACCTGATAGAAGTGCCAATTATGAGAAGCC
CTCCGAGCTTGGTCAGAGGGTTGAAGATCAGAAGGACTTCCCTACCACCG
TGGAGCATCAGTGGGGGTGTAAGTGATCCCAGCCCTTCTATTTGCTTCCT
CTCCAGCATTTCCCCCGTTTCCCGAGGGGCATCCAGCCGTGTTGCCTGGG
GAGGACCCACCCCCCTATTCACCCTTAACTAGCCCGGACAGTGGGAGTGC
CCCTATGATCACCTGCCGAGTCTGCCAATCTCTCATCAACGTGGAAGGCA
AGATGCATCAGCATGTAGTCAAATGTGGTGTCTGCAATGAAGCCACCGTG
AGTTACACATATCTATGAAATGGGCCCTGTTTCCTGGATCCTCTTTCTGA
TGTCTTGGTTCTAGACCCTGACCTTCCGGCTATTAGCCAAGTGCTTTTGA
TGATACCCAGGTTTCAGTTCCAGGTGTCTCACACAGCCATTTCCCCAGAA
GCCACTCACCAAAGCTAATGTTCACTTTCTCTCACTTTTACACCTAGCCT
AGTTCCTATTTGCAAATCTCATGATATAGTCTTTCTTTTATTTCTCCTTC
CTGGTTAGCACCTTATTTTTCTGATCTCATAAAGTGTTTTTGGAGGGAAG
TGGAGGGGATTGGGATTAGAGGTTTGCTTGCTGATGACCCTATTATTCTC
TAGCCAATCAAGAATGCACCCCCAGGGAAAAAATATGTTCGATGCCCCTG
TAACTGTCTCCTTATCTGCAAAGTGACATCCCAACGGATTGCATGCCCTC
GTCCCTACTGGTAAGAGGCATAAGGTGGGGAAGGGCCTAAGTGGGGAACT
GGAAAGTCAAAAAAGGATGAGCGTATACAGAGAATGTAAAGGTGAGAGAG
CCTAGTGTTTATTTAGGAGAAAAGGCTTTGAAGCATGTGCCTCAGGAATG
TTATAGCTGTCTTTCTCGTTTCTCAATAAAAATATTGAGATGAAATGATG
TCGTTTCGGAGAATAGAGAGCCTTGGGGACTGGGTGTGTTATCCTGAGGT
CGGAGGGGAATTGGGGACCTGAAGTTTAAACAGTGCTCTTTCTTTCTCAA
GGATTCTTGAGGGTATACAGTTGGGGGACAGAGTATCTTAAGTACAGAGA
AGTCGAGTGACTTAATAGACAGGGAGTGGGGGATGTGGAACAGGGACTGT
GAAGATTTTTAGGATTAAAAATTTTTCAAACACAAGTTTGAAAATACAAG
TCTTTTTCTTTTGTATAGCAAAAGAATCATCAACCTGGGGCCTGTGCATC
CCGGACCTCTGAGTCCAGAACCCCAACCCATGGGTGTCAGGGTTATCTGT
GGACATTGCAAGAATACTTTTCTGGTGAGGAAGGGGTATTGGGAAGGGGA
GGGGAAAGGAGACTAAGAGTCATTTCGAGTATATTTCTTAGAGTAATGGT
AATGACCCCTGAAAGGTCTGTCCTATGGGAACATGTTCTGCATCCCCACC
CCAAGGTTCTCATTGAGGGAGACCCTGCTTGTGCTATTATTTTTGTTTTC
TTTCTCCATAGTGGACAGAGTTCACAGACCGCACTTTGGCACGTTGTCCT
CACTGCAGGAAAGTGTCATCTATTGGGCGCAGATACCCACGTAAGAGATG
TATCTGCTGCTTCTTGCTTGGCTTGCTTTTGGCAGTCACTGCCACTGGCC
TTGCCGTGAGTACCCTTGCCCCAACCTCTTTCATTCTGCAGCCTCATCTC
CATAGGCTAAGATTTGGGAAACTGCTACCCTAAAAAAAAGTGGAAGAAAC
TTAGGGGACTAGTTTGTTTTGTTTTAAGATATGGATGAGCTAAAGTGCAA
AGTGGCTGATCAAACAGACTTTATTACTACTACAAGAGTGAAAAACAGCC
TTCCTTTCTCTGTAGGATGAGGATAGGACAGTGAAATTCTTAATTTAAGA
GTTGCTATTTTTCAAACCTGGCTCAGTTGTCAGATATTAAGAAAAACTGA
GATACAGTGTGGGATGGGATGAGTATGTTACGCCTAAGGGAAGGAAGCTG
ATCAGCTCTGCCTTTAAGAAGGTCCCTGAGGGTGGCTACATGTGGATAAG
GAACAAGGACTGAAGCGTGAGTTATTACTGTTCTTAGAACTAATAGGAGG
TAGTGGAGACCAACATTAACCCCATCTTTCTTTTCTTCTCCCTCCTTATC
TTCATCAGTTTGGCACATGGAAGCATGCACGGCGATATGGAGGCATCTAT
GCAGCCTGGGCATTTGTCATCCTGTTGGCTGTGCTGTGTTTGGGCCGGGC
TCTTTATTGGGCCTGTATGAAGGTCAGCCACCCTGTCCAGAACTTCTCCT
GAGCCTGATGACCCACAGACTGTGCCTGGCCCCTCCCTGGTGGGGACAGT
GACACTACGAAGGGAGCTGGGGTAGTTAAAGGCTCCCGGGGCTTCTAGAA
GGAAGCCAAGCAGCTGCCTTCCTTTTCCCTGGGGAGAGGTAGGAAGGAAC
CAGGCCCTCACTTAGGTTTGGAGGGGCAGATAAGAGCACTGCTGACCATC
TGCTTTCCTCCAAGGGTTGCTGTGTCTAGGGTGAAGTAGGCAAAACGTTG
CCCTTAAAACTGGGCCCTGAAGACGGTTCCAGCCTTGTCCTTCCTGTGTG
CTCCCTGAGAGCCATTCCTGTCCCTTACACATTCCAGGGCAGGGTGGGGG
TGGGTAGCCCTGGGGGTTCCCCTCCCTCTTGTGCACCATTAGGACTTTGC
TGCTGCTATTGCACTTCACCAGAGGTTGGCTCTGGCCTCAGTACCCTCAG
TCTCCTCTCCCCACATTGTGTCCTGTGGGGGTGGGGTCAGCCGCTGCTCT
GTACAGAACCACAGGAACTGATGTGTATATAACTATTTAATGTGGGATAT
GTTCCCCTATTCCTGTATTTCCCTTAATTCCTCCTCCCGACCTTTTTTAC
CCCCCCAGTTGCAGTATTTAACTGGGCTGGGTAGGGTTGCTCAGTCTTTG
GGGGAGGTTAGGGACTTATCCTGTGCTTGTAAATAAATAAGGTCATGACT
CTAAAAAAAAAAAAAAAAAAAAAAAAAAAAAAAAAAA
>Hs.302144_mRNA_gi|11493400|gb|AF130047.1|AF130047
Homo sapiens clone FLB3020 polyA = 0
CTGTCAGCACGGGGCCTGGCATGTAATTGGTCTGCACCCACTGGTGCACT
GAACTGCCATAACCTCAGGTTTTCTTTCTTGCTGATACCCCTGGGTCATG
TTCTTTGGCAAATAACATGATTCATTATGAAGTAGAGTTCAGCAAAGGAC
AAGGATGAAAGTTGTCATTTAGAGAACTGCCATTCAGACTTTCTTGTCTA
GGTAAAGAGCAAGGTCTTCTCTCTTTTCAACTCATTTTCTAAATTTAAAC
TGACGATGAGAATATGGATGATGTGTAGCTTCCTTCTCCCCCACTGATTT
TTGGTTCAGGCTCTGGGTTTTTGGCAAGAACTTACAGATCTCACTTATTA
TTGGCCACCCTTCTGCTTTAAGACCTGTCAGGGCTTGTCTGAAATAAAAC
TGGAAGCACTTCTGATTCCATCCTCACTGCTTTCCTCCTTCACCGTCAGA
CAGCATTACTGTATAGCACTGAGTGAGGGGCCCTGACACTGGAAGGTGGC
AGGTGGGGCCTGGCCGCCAGTGAGGTATCATCATTTGTGTGTGCTCATGT
GTGCGTTGGGCTTGTTGTATCTGAGGCATGAACATTCCATATACACGGCT
TAAAGAGTTTTCTTCCCATACCGAAAGCATATATTCGGAGAGGACCCAAC
TTATTCAGCATAGCCTTGTTCCCATAGTAGCCATCCTATTCCCCCACAGC
CTCTACTTTAGGAAAGCTCCCCGTCCCCATATGAAATCCAAACCAAAAAA
GATATATCACTTTCAGCTCAATTATTCCATAATTACAAGATATTAGGCTA
GTGGGCTCTTTATTGGTTGGGTCTTATATTAATGTTATATGCTAGCCTTG
TAATTTTGAGCTCCTCTATGGATGTTAATTTTAGTGAAACTCTATATTGA
AGAAAAGATGGGACTAAGGGGGAGACAGGAGGAGGAAAGAAAGCAGAGAC
AGGCAAAGAATCATAGCCTGAAATTCAACAGCAAGCATGGCTTATGAAGA
TCAAGTTATATTTTTGCTTCATGAATCATTGTCAGACAAATTAAGAACAT
ATTGTTTCTTATTTATCTATTGTCAAGGATTCACTATCAGACACTAAGAA
TGAATCTTGATTTTCATAAGCTCTGTTGACACCATGGAGCCACAGAGCAT
AAAACTTGCATCTAATAAAGAAAGTGCAACATGGAACAGCAGGGAGTGGA
ATACCAGCACAACTCACAGCTGCTTCCTGTTCCTCGTCCCTGTTTTCAGG
AATGTTTCTTAGCAGGAAGTTTTTTAATAGACCGAGAATTTGTTATATGT
ATTCTAAGAAAAGTTGTAGTTGTAGATGCATTACTCTCCCAAATCTTAGA
GATCAGGGATGATTATGTTCCATTTTTGTTTGGTGAGTTCCCATCTTTGT
ATGTACCTCCTTGCTCCCGGCTGTCCTCCTCTCCTCTTCCCTAGTGAGTG
GTTAATGAGTGTTAATGCCTAAACCATACTTGTTTTATGGACACTTCTAT
AATGGATTCGTTGCATAATTTTCATGCAGTGTATAGTGTTACTAGTTGGA
AATTCTTGGAGGACTCTTAGCTGTCTGATGAAATTCCTAGTAGAAATTTT
TGTTTTGAATTCCTAAAGTTGAAATATGAAAATTATATTTTAATTTGATT
C
>Hs.26510_mRNA_2 gi|11345385|gb|AF308803.1|
AF308803 Homo sapiens chromosome 15 map 15q26
polyA = 3
AGTTTTTCTGGTAGAAGGCGGGGTTCTCCTCGTACGCTGCGGAGTCTCTG
CGGGGTGTAGACCGGAATCCTGCTGACGGGCAGAGTGGATCAGGGAGGGA
GGGTCGAGACACGGTGGCTGCAGGTCTGAGACAAGGCTGCTCCGAGGTAG
TAGCTCTCTTGCCTGGAGGTGGCCATTCATTCCTGGAGTGCTGCTGAGGA
GCGAGGGCCCATCTGGGGTCTCTGGAAGTCGGTGCCCAGGCCTGAAGGAT
AGCCCCCCTTGCGCTTCCCTGGGCTGCGGCCGGCCTTCTCAGAACGAAGG
GCGTCCTTCCACCCCGCGGCGCAGGTGACCGCTGCCATGGCTTTTCCCCA
TCGGCCGGACGCCCCTGAGCTGCCTGACTTCTCCATGCTGAAGAGGCTGG
CTCGAGACCAGCTCATCTATCTGCTGGAGCAGCTTCCTGGAAAAAAGGAT
TTATTCATTGAGGCAGATCTCATGAGCCCTTTGGATCGAATTGCCAATGT
CTCCATCCTGAAGCAACACGAAGTAGACAAGCTATACAAGGTGGAGAACA
AGCCAGCCCTCAGCTCCAATGAACAATTGTGCTTCTTGGTCAGACCCCGC
ATCAAGAATATGCGATACATTGCCAGTCTTGTCAATGCTGACAAATTGGC
TGGCCGAACTCGCAAATACAAAGTGATCTTCAGCCCTCAAAAGTTCTATG
CGTGTGAGATGGTGCTTGAGGAAGAGGGAATCTATGGAGATGTGAGCTGT
GATGAATGGGCCTTCTCTTTGCTGCCTCTTGATGTGGATCTGCTGAGCAT
GGAACTACCAGAATTTTTCAGGGATTACTTTCTGGAAGGAGATCAGCGTT
GGATCAACACTGTAGCTCAGGCCTTACACCTTCTCAGCACTCTCTATGGA
CCCTTTCCAAACTGCTATGGAATTGGCAGGTGCGCCAAGATGGCATATGA
ATTGTGGAGGAACCTGGAGGAGGAGGAGGATGGCGAAACCAAGGGCCGAA
GGCCAGAGATTGGACATATCTTTCTCTTGGACAGAGATGTGGACTTTGTG
ACAGCACTTTGCTCCCAAGTGGTTTATGAGGGCCTAGTAGATGACACCTT
CCGCATCAAGTGTGGGAGTGTCGACTTTGGCCCAGAAGTCACATCCTCTG
ACAAGAGCCTGAAGGTGCTACTCAATGCCGAGGACAAGGTGTTTAATGAG
ATTCGGAACGAGCACTTCTCCAATGTCTTTGGCTTCTTGAGCCAGAAGGC
CCGGAACTTGCAGGCCCAGTATGATCGCCGGAGAGGCATGGACATTAAGC
AGATGAAGAATTTCGTGTCCCAGGAGCTCAAGGGCCTGAAACAGGAGCAC
CGCCTGCTGAGTCTCCATATTGGGGCCTGTGAATCCATCATGAAGAAGAA
AACCAAGCAGGATTTCCAGGAGCTAATCAAGACTGAGCATGCACTGCTAG
AGGGGTTCAACATCCGGGAGAGCACCAGCTACATTGAGGAACACATAGAC
CGGCAGGTGTCGCCTATAGAAAGCCTGCGCCTCATGTGCCTTTTGTCCAT
CACTGAGAATGGTTTGATCCCCAAGGATTACCGATCTCTGAAAACACAGT
ATCTGCAGAGCTATGGCCCTGAGCACCTGCTAACCTTCTCCAATCTGCGA
AGAGCTGGGCTCCTAACGGAGCAGGCCCCCGGGGACACCCTCACAGCCGT
GGAGAGTAAAGTGAGCAAGCTGGTGACCGACAAGGCTGCAGGAAAGATTA
CTGATGCCTTCAGTTCTCTGGCCAAGAGGAGCAATTTTCGTGCCATCAGC
AAAAAGCTGAATTTGATCCCACGTGTGGACGGCGAGTATGATCTGAAAGT
GCCCCGAGACATGGCTTACGTCTTCAGTGGTGCTTATGTGCCCCTGAGCT
GCCGAATCATTGAGCAGGTGCTAGAGCGGCGAAGCTGGCAGGGCCTTGAT
GAGGTGGTACGGCTGCTCAACTGCAGTGACTTTGCATTCACAGATATGAC
TAAGGAAGACAAGGCTTCCAGTGAGTCCCTGCGCCTCATCTTGGTGGTGT
TCTTGGGTGGTTGTACATTCTCTGAGATCTCAGCCCTCCGGTTCCTGGGC
AGAGAGAAAGGCTACAGGTTCATTTTCCTGACGACAGCAGTCACAAACAG
CGCTCGCCTTATGGAGGCCATGAGTGAGGTGAAAGCCTGATGTTTTTCCC
GGCCAGTGTTGACATCTTCCCTGAACACATTCCTCAGTGAGATGCAGGCA
TCTGGCACCCAGCTGCTATAACCAAGTGTCCACCAACTACCTGCTAAGAG
CCGGGAGCATGGAACGTGTTGGGATTTAGAGAACATTATCTGAGAAAAGA
GTTCACTTCCTGCTCCCAGGATATTTCTCTTTTCTGTTTATGAAGTACAA
CCCATGCTGCTAAGATGCGAGCAGGAAGAGGCATCCTTTGCTAAATCCTG
TTTGAATGTCATTGTAAATAAAGCCTCTGCTCTCAGATGTAAAAAAAAAA
AAAAAAAAAAA
>Hs.324709_mRNA_2 gi|12655026|gb|BC001361.1|
BC001361 Homo sapiens clone MGC:2474 IMAGE:3050694
polyA = 2
GGCACGAGGGGTCGCGCTGCCGCCGTTTTATTTGAAGACATCGTCCAGTT
CTGACCATGGACTCGCAGCCATCGGCCCTTAGTTTCCATCCCCTCTAGTG
GGCCTTCGGGGGCTCTACTGACGTCCCTCCTTCCCTTGGTACCGGGCCGG
GGAAGTGTTCTCGGGCGCGGGAGGTTCCGCATGCCCAGGCCTGGCCAGGG
GAGATGACCGATCCGTCGCTGGGGCTGACAGTCCCCATGGCGCCGCCTCT
GGCCCCGCTCCCTCCCCGGGACCCAAACGGGGCGGGATCCGAGTGGAGAA
AGCCCGGGGCCGTGAGCTTCGCCGACGTGGCCGTGTACTTCTCCCGGGAG
GAGTGGGGCTGCCTGCGGCCCGCGCAGAGGGCCCTGTACCGGGACGTGAT
GCGGGAGACCTACGGCCACCTGGGCGCGCTCGGTGAGAGCCCCACCTGCT
TGCCTGGGCCCTGCGCCTCCACAGGCCCTGCCGCGCCTCTGGGAGCTGCG
TGTGGAGTTGGGGGCCCCGGGGCCGGGCAGGCGGCCTCCTCGCAGCGTGG
GGTTTGCGTTCTTCTCCCCCAGGAGTCGGAGGCAGCAAGCCGGCGCTCAT
CTCCTGGGTGGAGGAGAAGGCCGAACTGTGGGATCCGGCTGCCCAGGATC
CGGAGGTGGCGAAGTGTCCGACAGAAGCGGACCCAGCAGATTCCAGAAAC
AAGGAAGAGGAAAGACAAAGGGAAGGGACGGGAGCCCTGGAGAAGCCCGA
CCCTGTGGCCGCCGGGTCTCCTGGGCTGAAGGCTCCCCAAGCCCCCTTTG
CCGGGTTGGAGCAGCTGTCCAAGGCCCGGCGCCGGAGTCGCCCCCGCTTT
TTTGCCCACCCCCCTGTCCCCCGAGCTGACCAGCGTCACGGCTGCTACGT
GTGCGGGAAGAGCTTCGCCTGGCGCTCCACACTGGTGGAGCACATTTACA
GCCACAGGGGCGAGAAGCCCTTCCACTGCGCAGACTGCGGCAAGGGCTTC
GGCCACGCTTCCTCCCTGAGCAAACACCGGGCCATCCATCGTGGGGAGCG
GCCCCACCGCTGTCCCGAGTGTGGTCGGGCCTTCATGCGCCGCACGGCGC
TGACTTCTCACCTGCGCGTTCACACTGGCGAGAAGCCCTACCGCTGCCCG
CAGTGTGGCCGCTGCTTCGGCCTGAAGACCGGCATGGCCAAGCACCAATG
GGTCCATCGGCCCGGGGGCGAGGGGCGTAGGGGCCGGCGCCCTGGGGGGC
TGTCTGTGACCCTGACTCCTGTCCGCGGGGACCTGGACCCGCCTGTGGGC
TTCCAGCTGTATCCAGAGATATTCCAGGAATGTGGGTGACGGCCTAAAAA
GTGACCATCTAGACATTGTGGGCGGCCCGAGATGGGCTCAGGGGCCCGAA
CCTCTGCAGCGGCCTGCAGGGAGGTCCCAGAATCCACCGCAAGAGCTGGC
CTGGGGTGCGGACAGTCTGATCTTGGGCTCTCAGCAGCCTCTTCTGCCAG
CACCTTGCTCCCCGCTGCCCTGGGCTCTCCAAGGCCCCCTTTGCTGAGGC
AGGGCTGAGGTGAGAACCCCCCAGACCTCCATACAGGGAAGCAAAAGCTG
TTTCTCCTCCCAGAGATGCTAAGAGGATTGAGGTAGAGAAGAACCTTGTT
TTCTCTGTTGTCTTTTTCTTTTTACTTTTTTAATTTTTTGAGACGGAGTT
TTGCTCTTGTTGCCCAGGCTGGAGTGCAATGGTGCGATCTCGACTCACTG
CAACTTCCACCTCCTGGAGTCAAGCGATTCTCCTGCCTCAGCCACCCAAG
TAGCTGGAATTACAGGCACCTGCCACTATGCCCGGCTAACTTTTTGTATT
TTTAGTAGAGATGGGGTTTCACCATGTTGGCTAGGCTGGTCTCGAACTCC
TGCCCTCAGGTGATCCACCCACCTCTGCCTCCCAAAGTGCTGGGATTACA
GGCGTGAGCCACCTCACCTGGCCTTTTCTTTTTTATTCTTTGACCTTCCC
ACAAGACAATACCCATTGTCTGTTTTTTTTGTTTATTTATTTACTTATTA
AGACAGCATCTTGCTCCTCACCCAGGCTGGAATGCAGTGGTGTGAACTGG
GCTCACTGCAGCCTAGACCTGCTGGGCTCAAGGAATCCTCCTGCCCCAGC
CTCTCAGATGGCTGTGACTACAGGTGGGCAACACTATGCCTGGTTAATTT
TTAAATTTTTTTGCAGAGATGGGGTTCCCACTATGTTGATCAGGCTGGTC
TCAAACTCCTCGGTTCAAGCAATTCGCCCACCTTGGCCTCCCAAAGTGCT
GGGATTACAGGGGAGCCACTGCACTGGCCTTCATTGTCTTTTTGCTGCAC
AACCTAAAAAACCAGTGACCCTGTATTGGAAAAAAAAAAAAAAAAAAAAA
A
>Hs.65756_mRNA_3 gi|3641494|gb|AF035154.1|AF035154
Homo sapiens chromosome 16 map 16p13.3 polyA = 3
GCCATGGCCGCCGGCCCCGCGCCGCCCCCCGGCCGCCCCCGGGCGCAGAT
GCCGCATCTGAGGAAGGTGCGAGGCGGATGGAGCGGGTGGTCGTGAGCAT
GCAGGACCCCGACCAGGGCGTGAAGATGCGGAGCCAGCGCCTGCTGGTCA
CCGTCATTCCCCACGCGGTGACAGGCAGCGACGTCGTGCAGTGGTTGGCC
CAGAAGTTCTGCGTCTCGGAGGAGGAGGCCCTGCACCTGGGCGCCGTCCT
GGTGCAGCATGGCTACATCTACCCGCTGCGCGACCCCCGTAGCCTCATGC
TCCGGCCAGACGAGACGCCCTACAGGTTCCAGACCCCGTACTTCTGGACA
AGTACCCTGAGGCCGGCTGCAGAGCTGGACTATGCCATCTACCTGGCCAA
GAAGAACATCCGAAAACGGGGGACCCTGGTGGATTATGAGAAGGACTGCT
ATGACCGGCTACACAAGAAGATCAACCACGCATGGGACCTGGTGCTGATG
CAGGCGAGGGAGCAGCTGAGGGCAGCCAAGCAGCGCAGCAAGGGGGACAG
GCTGGTCATTGCGTGCCAGGAGCAGACCTACTGGCTGGTGAACAGGCCCC
CGCCCGGGGCCCCCGATGTGCTGGAGCAGGGTCCAGGGCGGGGATCCTGC
GCTGCCAGCCGTGTGCTCATGACCAAGAGTGCAGATTTCCATAAGCGGGA
GATCGAGTACTTCAGGAAAGCGCTGGGCAGGACCCGAGTGAAGTCCTCCG
TCTGCCTTGAGGCGTACCTGAGTTTCTGCGGCCAGCGTGGACCCCACGAT
CCCCTCGTGTCGGGGTGCCTGCCCAGCAATCCCTGGATCTCAGACAATGA
CGCCTACTGGGTCATGAATGCCCCCACGGTGGCTGCCCCCACGAAGCTCC
GTGTGGAGAGATGGGGCTTCAGCTTCCGGGAGCTCCTGGAGGACCCCGTG
GGGCGGGCCCACTTCATGGACTTTCTGGGAAAGGAGTTCAGTGGAGAAAA
CCTCAGCTTCTGGGAGGCATGTGAGGAGCTTCGATATGGAGCGCAGGCCC
AGGTCCCCACCCTGGTGGATGCCGTGTACGAGCAGTTCCTGGCCCCCGGA
GCTGCCCACTGGGTCAACATCGACAGCCGGACCATGGAGCAGACCCTGGA
GGGGCTGCGCCAGCCCCACCGCTATGTCCTGGATGACGCCCAGCTGCACA
TATACATGCTCATGAAGAAGGACTCCTACCCAAGGTTCCTGAAGTCTGAC
ATGTACAAGGCCCTCCTGGCAGAGGCTGGGATCCCGCTGGAGATGAAGAG
ACGCGTGTTCCCGTTTACGTGGAGGCCACGGCACTCGAGCCCCAGCCCTG
CACTCCTTCCCACCCCTGTGGAGCCCACAGCGGCTTGTGGCCCTGGGGGT
GGAGATGGGGTGGCCTAGTGGACCTGGCCCATCTGCCACTCTAGTCCCTG
CAGCTCAACGTCCTGCGTGAATGCAGCAGCCACCCCCGTCTTGGCCCAGG
TCCTGGGGGCTGCTGAACCCAGCACCAGTGTCCCCTTGTGCCCAGGGGGC
CCAGTCTTCTGTGGGGTGCACAGCCTCCCTCCCTCCAGCAAGCCCTCCCT
GCCCAGAAGGAATGGGTCCAGGTGTGGATTCCCAGGGAGGGGGTTCATTG
GCTCAGCTTGGGTCAGGGCAGAGCCTGTTACCTGAAGAGAGGTGAGACCA
AGGCCACAGGGAGCTCCACCTTCTCTGGTCTTCAGTCCAGCACTGGGTGC
CCATCCCCATCTCTAAAACCAGTAAATCAGCCAGCGAATACCCGGAAGCA
AGATGCACAGGCGGGCGGCTTCCCACACACCCGTCACAAGACGCGGACAT
GCAGGTCTCGGCGCGAGCTCTGCCCCGTCCAAGAGCCTCTCCGCTGTCGC
CCAGTGTGAGCCTGGAAGAGGACCCAAGAGAGTGCCGTGCTGAGGCTGCC
TCGAGGTCACTGCCTTCCGGAGCTGCGCCTATTCCTCCCTCGCCAAACGC
GTTCCAGAATTTGTCCACAGGTGCGCCGGCACCTGCTTTCCCACCTCGAG
GCCGCGGCCTCCCCCCCGATTTATAGACAACTCTGACATTGTCACCCCAC
TGACGAGGCCCGATTCCATAGGGTGGATCCTTGCCAGGCGTCCCTGATCC
TCCCTGCCCAAGTCTTCCTTCGTGAGCTGGCCTTGCTCCCCATCCCCCAA
GTGCCTCACCAGTCCCCCAGACTGGGTGAAGGTACAGCTGGCTCCTTTCG
GGGGTGCAGCTTCAACTCTCTCGGCGGTAGGGCGGTGCCATCCCCACCCA
TAGGGCTGGCTCACATCCAGTCACTCCCAACAGCGTCCAGCACACAAATA
AAAGACCCTTGGGCCCTGGCTCTGAGAAAAAAAA
>Hs.165743_mRNA_2 gi|13543889|gb|BC006091.1|
BC006091 Homo sapiens clone MGC:12673
IMAGE:3677524 polyA = 3
AGACTGCCGAGCAGCCTTGAGCCGTTGAGCAGCTGAACAGAGGCCATGCC
GGGGCACTCCGAGGCCTGAGACGACCACGCCTGTGCCGCTGAGGACCTTC
ATCAGGGCTCCGTCCACTTGGCCCGCTTGGCTGTCCAATCACACTCCAGT
GTCAACCACTGGCACCCAGCAGCCAAGAGAGGTGTGGCGTGGCCCTGGGG
ACGCATGGCTGAGGCAGGAACAGGTGAGCCGTCCCCCAGCGTGGAGGGCG
AACACGGGACGGAGTATGACACGCTGCCTTCCGACACAGTCTCCCTCAGT
GACTCGGACTCTGACCTCAGCTTGCCCGGTGGTGCTGAAGTGGAAGCACT
GTCCCCGATGGGGCTGCCTGGGGAGGAGGATTCAGGTCCTGATGAGCCGC
CCTCACCCCCGTCAGGCCTCCTCCCAGCCACGGTGCAGCCATTCCATCTG
AGAGGCATGAGCTCCACCTTCTCCCAGCGCAGCCGTGACATCTTTGACTG
CCTGGAGGGGGCGGCCAGACGGGCTCCATCCTCTGTGGCCCACACCAGCA
TGAGTGACAACGGAGGCTTCAAGCGGCCCCTAGCGCCCTCAGGCCGGTCT
CCAGTGGAAGGCCTGGGCAGGGCCCATCGGAGCCCTGCCTCACCAAGGGT
GCCTCCGGTCCCCGACTACGTGGCACACCCCGAGCGCTGGACCAAGTACA
GCCTGGAAGATGTGACCGAGGTCAGCGAGCAGAGCAATCAGGCCACCGCC
CTGGCCTTCCTGGGCTCCCAGAGCCTGGCTGCCCCCACTGACTGCGTGTC
CTCCTTCAACCAGGATCCCTCCAGCTGTGGGGAGGGGAGGGTCATCTTCA
CCAAACCAGTCCGAGGGGTCGAAGCCAGACACGAGAGGAAGAGGGTCCTG
GGGAAGGTGGGAGAGCCAGGCAGGGGCGGCCTTGGGAATCCTGCCACAGA
CAGGGGCGAGGGCCCTGTGGAGCTGGCCCATCTGGCCGGGCCCGGGAGCC
CAGAGGCTGAGGAGTGGGGCAGCCCCCATGGAGGCCTGCAGGAGGTGGAG
GCACTGTCAGGGTCTGTCCACAGTGGGTCTGTGCCAGGTCTCCCGCCGGT
GGAAACTGTTGGCTTCCATGGCAGCAGGAAGCGGAGTCGAGACCACTTCC
GGAACAAGAGCAGCAGCCCCGAGGACCCAGGTGCTGAGGTCTGAGAGGGA
GATGGCCCAGCCTGACCCCACTGGCCACTGCCATCCTGCTGCCTTCCCAG
TGGGGCTGGTCAGGGGGCAGCCTGGCCACTGCCTAGCTGGAATGGGAGGA
AGCCTGCAGGTGGCACCGGTGGCCCTGGCTGCAGTTCTGGGCAGCATCCT
CCCAAGCAGAGACCTTGCTGAAGCTCCTGGGGTGTGGGGTGTGGGCTGGA
AGCACTGGCTCCCTGGTAGGGACAATAAAGGTTTTGGGTCTTTCAAAAAA
AAAAAAAAAAAAAAAAAAAAAAAAAAAAAAAAAAAAAAAAAAAAAAAAAC

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of determining the expression level of one or more reference gene sequences in a cell, said method comprising
determining the expression level(s) of one or more gene sequences which comprises or hybridizes to at least 20 nucleotides of one or more of SEQ ID NOs: 1-16.

2. A method of determining the expression level of one or more gene sequences of interest in a cell of a sample, said method comprising
determining the expression level(s) of said one or more gene sequences in said cell; and
comparing said expression level(s) to the expression level of a reference gene sequence determined by the method of claim 1.

3. The method of claim 1, further comprising determining the expression levels of other transcribed sequences, in addition to said reference gene sequence(s).

4. The method of claim 2, further comprising determining the expression levels of other transcribed sequences, in addition to said reference gene sequence(s) or said gene sequences of interest.

5. The method of claim 1 wherein said expression levels are determined by use of a microarray.

6. The method of claim 2 wherein the expression levels of said one or more gene sequences of interest comprises measuring all or part of the expressed sequences.

7. The method of claim 1 wherein said expression level(s) are determined by amplification of all or part of the transcribed sequences, or
reverse transcription and labeling RNA corresponding to said transcribed sequences.

8. The method of claim 7 wherein said amplification comprises linear RNA amplification or quantitative PCR.

9. The method of claim 7 wherein said amplification is of sequences present within 300 nucleotides of the polyadenylation sites of the transcripts.

10. The method of claim 7 wherein said amplification is quantitative PCR amplification of at least 50 nucleotides of the transcripts.

11. The method of claim 1 wherein said cell is in an FFPE sample.

12. The method of claim 1 wherein said cell is a tumor cell.

13. The method of claim 12 wherein said cell is a tumor cell of a type selected from adrenal gland, brain, breast, carcinoid-intestine, cervix-adenocarcinoma, cervix-squamous, endometrium, gall bladder, germ cell-ovary, GIST, kidney, leiomyosarcoma, liver, lung-adenocarcinoma-large cell, lung-small cell, lung-squamous, lymphoma-B cell, lymphoma-Hodgkin's, lymphoma-T cell, meningioma, mesothelioma, osteosarcoma, ovary-clear cell, ovary-serous, pancreas, prostate, skin-basal cell, skin-melanoma, skin-squamous, small and large bowel, soft tissue-liposarcoma, soft tissue-MFH, soft tissue-sarcoma-synovial, stomach-adenocarcinoma, testis-other (or non-seminoma), testis-seminoma, thyroid-follicular-papillary, thyroid-medullary, and urinary bladder.

14. A microarray comprising oligonucleotide probes to detect the expression of a reference gene sequence which comprises or hybridizes to at least 20 nucleotides of one or more of SEQ ID NOs: 1-16.

15. The method of claim 1 wherein expression of all 8 genes corresponding to SEQ ID NOs: 1-16 is determined.
